(19) United States
(12) Patent Application Publication
(10) Pub. No.: US 2020/0284810 A1
(43) Pub. Date: Sep. 10, 2020

(54) METHODS FOR DETECTION OF FOLATE RECEPTOR 1 IN A PATIENT SAMPLE

(71) Applicant: ImmunoGen, Inc., Waltham, MA (US)

(72) Inventors: Raymond XU, Acton, MA (US); Kerry CULM-MERDEK, Natick, MA (US)

(21) Appl. No.: 16/644,695

(22) PCT Filed: Sep. 5, 2018
(86) PCT No.: PCT/US2018/049529

§ 371 Date: Mar. 5, 2020

Related U.S. Application Data
(60) Provisional application No. 62/554,532, filed on Sep. 5, 2017.

(51) Int. Cl. G01N 33/82; G01N 33/68; G01N 33/543; C07K 16/18

(52) U.S. Cl. CPC G01N 33/82; G01N 33/6848; C07K 2317/92; C07K 16/18; C07K 2317/565; G01N 33/54326

(57) ABSTRACT

The invention generally relates to methods and kits for the detection of human folate receptor 1 in a sample. Peptides of human folate receptor 1 are further provided.

### FIELD OF THE INVENTION

The field of this invention generally relates to a methods and kits for detecting human folate receptor 1 (FOLR1) in a sample.

### BACKGROUND OF THE INVENTION

Cancer is one of the leading causes of death in the developed world, with over one million people diagnosed with cancer and 500,000 deaths per year in the United States alone. Overall it is estimated that more than 1 in 3 people will develop some form of cancer during their lifetime. There are more than 200 different types of cancer, four of which—breast, lung, colorectal, and prostate—account for over half of all new cases (Jemal et al., 2003, Cancer J. Clin. 53:5-26).

Folate Receptor 1 (FOLR1), also known as Folate Receptor-alpha or Folate Binding Protein, is an N-glycosylated protein expressed on plasma membrane of cells. FOLR1 has a high affinity for folic acid and for several reduced folic acid derivatives. FOLR1 mediates delivery of the physiological folate, 5-methyltetrahydrofolate, to the interior of cells.

FOLR1 is overexpressed in the vast majority of ovarian cancers, as well as in many uterine, endometrial, pancreatic, renal, lung, and breast cancers, while the expression of FOLR1 on normal tissues is restricted to the apical membrane of epithelial cells in the kidney proximal tubules, alveolar pneumocytes of the lung, bladder, testes, choroid plexus, and thyroid (Weitman S D, et al., Cancer Res 52: 3396-3401 (1992); Antony A C, Annu Rev Nutr 16: 501-521 (1996); Kalli K R, et al. Gynecol Oncol 108: 619-626 (2008)). This expression pattern of FOLR1 makes it a desirable target for FOLR1-directed cancer therapy.

Because ovarian cancer is typically asymptomatic until advanced stage, it is often diagnosed at a late stage and has poor prognosis when treated with currently available procedures, typically chemotherapeutic drugs after surgical de-bulking (von Gruenigen V et al., Cancer 112: 2221-2227 (2008); Ayhan A et al., Am J Obstet Gynecol 196: 81 e81-86 (2007); Harry V N et al., Obstet Gynecol Sury 64: 548-560 (2009)). Thus there is a clear unmet medical need for more effective therapeutics for ovarian cancers.

Some previous assays used to detect shed FOLR1 are not sufficiently specific to FOLR1. For example, some assays do not distinguish between FOLR1 and other folate receptor family members (FOLR2, 3, & 4) or report values for total FBP (Folate Binding Protein). Additionally, some assays require that human samples (e.g., plasma) be pre-treated with a light acid wash step to dissociate folic acid from the receptor. Some assay results may also have inaccuracies due to competitive effects between the antibody therapy and diagnostic antibody. Additionally, many commercially available kits are traditionally unreliable both in their reagents, and in their lot-to-lot stability. Evaluations of these kits have given very mixed results, and are intended for research use only. Many require that the human sample be pre-diluted before analysis to reduce the chance of false positives due to the “matrix effect.” Moreover, many current assays, e.g., ELISA-based assays, do not provide enough sensitivity to distinguish between variations in shed FOLR1 at physiological levels and are limited by false positive results. Thus, there is a clear need for highly sensitive and accurate methods to detect FOLR1 in patient samples.

### SUMMARY OF THE INVENTION

The present invention provides methods for detection of FOLR1 in a sample and can be used, for example, to quantitate the level of human FOLR1 in a sample.

In one embodiment, a method of detecting human folate receptor 1 (FOLR1) in a sample comprises: (a) capturing said folate receptor 1 (FOLR1) with an immunocapture reagent bound to a solid support; (b) eluting FOLR1 from the solid support; (c) digesting the eluted FOLR1; and (d) performing liquid chromatography-mass spectrometry (LC/MS) analysis on the digested FOLR1, wherein said FOLR1 is detected by monitoring the chromatographic separation and mass spectrometric response of at least one signature FOLR1 peptide.

In one embodiment, the level of FOLR1 in the sample is quantitated by LC/MS analysis. In one embodiment, the level of FOLR1 in the sample is quantitated by comparing the level of FOLR1 in the sample to a reference level of FOLR1.

In one embodiment, the immunocapture reagent comprises an antibody or antigen-binding fragment which binds to FOLR1. In one embodiment, binding of the antibody or antigen-binding fragment to FOLR1 is not competitively inhibited by binding of IMGN853 to FOLR1. In one embodiment, binding of the antibody or antigen-binding fragment to FOLR1 is not competitively inhibited by binding of huMov19 to FOLR1. In one embodiment, binding of the antibody or antigen-binding fragment to FOLR1 is not competitively inhibited by binding of a second antibody or antigen-binding fragment to FOLR1, wherein the second antibody or antigen-binding fragment comprises: a variable light chain (VL) complementarity determining region (CDR)-1 of SEQ ID NO: 59; a VL CDR-2 of SEQ ID NO: 60; a VL CDR-3 of SEQ ID NO: 61; a variable heavy chain (VH) CDR-1 of SEQ ID NO: 62; a VH CDR-2 of SEQ ID NO: 64; and a VH CDR-3 of SEQ ID NO: 65. In one embodiment, binding of the antibody or antigen-binding fragment to FOLR1 is not competitively inhibited by binding of a second antibody or antigen-binding fragment to FOLR1, wherein the second antibody or antigen-binding fragment comprises a variable heavy chain (VH) having the sequence of SEQ ID NO: 56 and a variable light chain (VL) having the sequence of SEQ ID NO: 57 or SEQ ID NO: 58. In one embodiment, binding of the antibody or antigen-binding fragment to FOLR1 is not competitively inhibited by binding of a second antibody or antigen-binding fragment to FOLR1, wherein the second antibody or antigen-binding fragment comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774. In one embodiment, binding of the antibody or antigen-binding fragment to FOLR1 is not inhibited by binding of folic acid to FOLR1.

In one embodiment, the antibody is muFR1-9. In one embodiment, the antibody is muFR1-13. In one embodiment, the antibody or antigen-binding fragment comprises a variable heavy chain (VH) complementarity determining region (CDR)-1 of SEQ ID NO: 1; a VH CDR-2 of SEQ ID NO: 2; a VH CDR-3 of SEQ ID NO: 3; a variable light chain (VL) complementarity determining region (CDR)-1 of SEQ ID NO: 13; a VL CDR-2 of SEQ ID NO: 14, and a VL CDR-3 of SEQ ID NO: 15. In one embodiment, the antibody or antigen-binding fragment comprises a variable heavy chain (VH) complementarity determining region (CDR)-1 of SEQ ID NO: 4; a VH CDR-2 of SEQ ID NO: 5; a VH CDR-3 of SEQ ID NO: 6; a variable light chain (VL) complementarity determining region (CDR)-1 of SEQ ID NO: 16; a VL CDR-2 of SEQ ID NO: 17, and a VL CDR-3 of SEQ ID NO: 18.

In one embodiment, the antibody or antigen-binding fragment comprises a variable heavy chain (VH) having at least 90% identity to the sequence of SEQ ID NO: 25 and a variable light chain (VL) having at least 90% identity to the sequence of SEQ ID NO: 29. In one embodiment, the antibody or antigen-binding fragment comprises a variable heavy chain (VH) having at least 95% identity to the sequence of SEQ ID NO: 25 and a variable light chain (VL) having at least 95% identity to the sequence of SEQ ID NO: 29. In one embodiment, the antibody or antigen-binding fragment comprises a variable heavy chain (VH) having at least 90% identity to the sequence of SEQ ID NO: 26 and a variable light chain (VL) having at least 90% identity to the sequence of SEQ ID NO: 30. In one embodiment, the antibody or antigen-binding fragment comprises a variable heavy chain (VH) having at least 95% identity to the sequence of SEQ ID NO: 26 and a variable light chain (VL) having at least 95% identity to the sequence of SEQ ID NO: 30.

In one embodiment, the antibody or antigen-binding fragment comprises a variable heavy chain (VH) having the sequence of SEQ ID NO: 25 and a variable light chain (VL) having the sequence of SEQ ID NO: 29. In one embodiment, the antibody or antigen-binding fragment comprises a variable heavy chain (VH) having the sequence of SEQ ID NO: 26 and a variable light chain (VL) having the sequence of SEQ ID NO: 30. In one embodiment, the antibody or antigen-binding fragment comprises a heavy chain having the sequence of SEQ ID NO: 33 and a light chain having the sequence of SEQ ID NO: 37. In one embodiment, the antibody or antigen-binding fragment comprises a heavy chain having the sequence of SEQ ID NO: 34 and a light chain having the sequence of SEQ ID NO: 38.

In one embodiment, the antibody or antigen-binding fragment binds to human FOLR1 with a Kd of about 1.0 nM to about 10 nM. In one embodiment, the antibody or antigen-binding fragment binds to human FOLR1 with a Kd of about 0.5 nM to about 5 nM.

In one embodiment, the solid support comprises a mass spectrometric immunoassay (MSIA) microcolumn. In one embodiment, the solid support comprises magnetic beads.

In one embodiment, at least one wash step is performed prior to eluting FOLR1 from the solid support. In one embodiment, two or more wash steps are performed prior to eluting FOLR1 from the solid support. In one embodiment, the wash step comprises contacting the FOLR1 bound to the solid support with washing buffers, a salt solution, and a detergent. In one embodiment, FOLR1 is eluted from the solid support with an acidic solution.

In one embodiment, the FOLR1 is reduced and alkylated prior to digesting the FOLR1. In one embodiment, FOLR1 is digested with Trypsin/Lys-C. In one embodiment, digesting the FOLR1 produces a peptide comprising the sequence of SEQ ID NO: 42. In one embodiment, digesting the FOLR1 produces a peptide comprising the sequence of SEQ ID NO: 43. In one embodiment, digesting the FOLR1 produces a peptide comprising the sequence of SEQ ID NO: 44. In one embodiment, digesting the FOLR1 produces a peptide comprising the sequence of SEQ ID NO: 45.

In one embodiment, at least two signature peptides of FOLR1 are selected and monitored at the LC/MS analysis step. In one embodiment, at least three signature peptides of FOLR1 are selected and monitored at the LC/MS analysis step. In one embodiment, at least four signature peptides of FOLR1 are selected and monitored at the LC/MS analysis step.

In one embodiment, the signature peptides comprise: (a) a peptide comprising the sequence of SEQ ID NO: 42; (b) a peptide comprising the sequence of SEQ ID NO: 43; (c) a peptide comprising the sequence of SEQ ID NO: 44; and (d) a peptide comprising the sequence of SEQ ID NO: 45.

In one embodiment, the sample comprises a bodily fluid. In one embodiment, the bodily fluid is plasma. In one embodiment, the bodily fluid is serum. In one embodiment, the bodily fluid is ascites fluid.

In one embodiment, the sample comprises a peripheral blood sample.

In one embodiment, the sample is obtained from a patient having cancer. In one embodiment, the cancer is selected from the group consisting of: ovarian, brain, breast, uterine, endometrial, pancreatic, renal, lung cancer, and cancer of the peritoneum. In one embodiment, the cancer is ovarian cancer. In one embodiment, detecting FOLR1 is not inhibited by IMGN853 present in the sample. In one embodiment, detecting FOLR1 is not inhibited by huMov19 present in the sample.

In one embodiment, detecting FOLR1 is not inhibited by an antibody or antigen-binding fragment present in the sample, wherein said antibody or antigen-binding fragment comprises: a variable light chain (VL) complementarity determining region (CDR)-1 of SEQ ID NO: 59; a VL CDR-2 of SEQ ID NO: 60; a VL CDR-3 of SEQ ID NO: 61; a variable heavy chain (VH) CDR-1 of SEQ ID NO: 62; a VH CDR-2 of SEQ ID NO: 64; and a VH CDR-3 of SEQ ID NO: 65. In one embodiment, detecting FOLR1 is not inhibited by an antibody or antigen-binding fragment present in the sample, wherein said antibody or antigen-binding fragment comprises a variable heavy chain (VH) having the sequence of SEQ ID NO: 56 and a variable light chain (VL) having the sequence of SEQ ID NO: 57 or SEQ ID NO: 58. In one embodiment, detecting FOLR1 is not inhibited by an antibody or antigen-binding fragment present in the sample, wherein said antibody or antigen-binding fragment comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774.

In one embodiment, detecting FOLR1 is not inhibited by folic acid present in the sample.

In one embodiment, the method can detect at least 0.5 ng/mL FOLR1 in a sample. In one embodiment, the method can detect at least 0.3 ng/mL FOLR1 in a sample. In one embodiment, the method can detect at least 0.25 ng/mL FOLR1 in a sample.

In one embodiment, the signal-to-noise ratio is at least 5. In one embodiment, the signal-to-noise ratio is at least 10.

In one embodiment, the FOLR1 is shed FOLR1.

The present invention also provides novel peptides of FOLR1. In one embodiment, a peptide consists of the sequence of SEQ ID NO: 42. In one embodiment, a peptide consists of the sequence of SEQ ID NO: 43. In one embodiment, a peptide consists of the sequence of SEQ ID NO: 44. In one embodiment, a peptide consists of the sequence of SEQ ID NO: 45.

The present invention also provides kits for the detection of FOLR1 in a sample. In one embodiment, the kit comprises: an immunocapture reagent which binds to FOLR1, a digestion reagent, and at least one peptide selected from the group consisting of: a) a peptide comprising the sequence of SEQ ID NO: 42; b) a peptide comprising the sequence of SEQ ID NO: 43; c) a peptide comprising the sequence of SEQ ID NO: 44; and d) a peptide comprising the sequence of SEQ ID NO: 45.

In one embodiment, the immunocapture reagent comprises an antibody or antigen-binding fragment which binds to FOLR1. In one embodiment, the antibody is muFR1-9. In one embodiment, the antibody is muFR1-13.

In one embodiment, the antibody or antigen-binding fragment comprises a variable heavy chain (VH) complementarity determining region (CDR)-1 of SEQ ID NO: 1; a VH CDR-2 of SEQ ID NO: 2; a VH CDR-3 of SEQ ID NO: 3; a variable light chain (VL) complementarity determining region (CDR)-1 of SEQ ID NO: 13; a VL CDR-2 of SEQ ID NO: 14, and a VL CDR-3 of SEQ ID NO: 15. In one embodiment, the antibody or antigen-binding fragment comprises the variable heavy chain (VH) complementarity determining region (CDR)-1 of SEQ ID NO: 4; a VH CDR-2 of SEQ ID NO: 5; a VH CDR-3 of SEQ ID NO: 6; a variable light chain (VL) complementarity determining region (CDR)-1 of SEQ ID NO: 16; a VL CDR-2 of SEQ ID NO: 17, and a VL CDR-3 of SEQ ID NO: 18.

In one embodiment, the antibody or antigen-binding fragment comprises a variable heavy chain (VH) having at least 90% identity to the sequence of SEQ ID NO: 25 and a variable light chain (VL) having at least 90% identity to the sequence of SEQ ID NO: 29. In one embodiment, the antibody or antigen-binding fragment comprises a variable heavy chain (VH) having at least 95% identity to the sequence of SEQ ID NO: 25 and a variable light chain (VL) having at least 95% identity to the sequence of SEQ ID NO: 29. In one embodiment, the antibody or antigen-binding fragment comprises a variable heavy chain (VH) having at least 90% identity to the sequence of SEQ ID NO: 26 and a variable light chain (VL) having at least 90% identity to the sequence of SEQ ID NO: 30. In one embodiment, the antibody or antigen-binding fragment comprises a variable heavy chain (VH) having at least 95% identity to the sequence of SEQ ID NO: 26 and a variable light chain (VL) having at least 95% identity to the sequence of SEQ ID NO: 30.

In one embodiment, the antibody or antigen-binding fragment comprises a variable heavy chain (VH) having the sequence of SEQ ID NO: 25 and a variable light chain (VL) having the sequence of SEQ ID NO: 29. In one embodiment, the antibody or antigen-binding fragment comprises a variable heavy chain (VH) having the sequence of SEQ ID NO: 26 and a variable light chain (VL) having the sequence of SEQ ID NO: 30.

In one embodiment, the antibody or antigen-binding fragment comprises a heavy chain having the sequence of SEQ ID NO: 33 and a light chain having the sequence of SEQ ID NO: 37. In one embodiment, the antibody or antigen-binding fragment comprises a heavy chain having the sequence of SEQ ID NO: 34 and a light chain having the sequence of SEQ ID NO: 38.

In one embodiment, the antibody or antigen-binding fragment binds to human FOLR1 with a Kd of about 1.0 nM to about 10 nM. In one embodiment, the antibody or antigen-binding fragment binds to human FOLR1 with a Kd of about 0.5 nM to about 5 nM.

### BRIEF DESCRIPTIONS OF THE DRAWINGS

FIG. 1 shows a schematic representation of FOLR1 immunocapture-LC/MS assay.

FIG. 2 shows an extracted ion chromatogram from a sample containing a low level of soluble FOLR1 in a surrogate matrix at 0.3 ng/mL.

FIG. 3 shows an extracted ion chromatogram from a normal human plasma sample.

FIG. 4 shows an extracted ion chromatogram from a patient pre-dose sample with elevated soluble FOLR1.

### DETAILED DESCRIPTION OF THE INVENTION

The present invention provides a novel method of detecting human folate receptor 1 (FOLR1) in a patient sample. An initial immunocapture step of FOLR1 is performed, followed by digestion of the captured FOLR1 into peptides and quantitative analysis of the peptides via liquid chromatography-mass spectrometry (LC/MS). Antibodies that do not competitively inhibit the binding of an anti-FOLR1 active agent (e.g., an active agent comprising the antibody huMov19, such as IMGN853) to FOLR1 are useful in the immunocapture step of the invention. Antibodies that do not competitively inhibit the binding of an anti-FOLR1 active agent are especially useful in capturing FOLR1 (e.g., shed FOLR1) in samples from patients who have been treated with the anti-FOLR1 active agent. Peptides of FOLR1 produced by the methods of the invention are also disclosed. Such peptides are useful in detecting the level of FOLR1 in a patient sample by LC/MS. Kits comprising the FOLR1-binding agents and FOLR1 peptides are also provided.

### I. Definitions

To facilitate an understanding of the present invention, a number of terms and phrases are defined below.

The terms “human folate receptor 1,” “FOLR1” or “folate receptor alpha (FR-α),” as used herein, refer to any native human FOLR1, unless otherwise indicated. Thus, all of these terms can refer to either a protein or nucleic acid sequence as indicated herein. The term “FOLR1” encompasses “full-length,” unprocessed FOLR1 as well as any form of FOLR1 that results from processing within the cell. The term also encompasses naturally occurring variants of FOLR1, e.g., splice variants (except those variants that encompass FOLR2, FOLR3, or FOLR4), allelic variants and isoforms. The FOLR1 polypeptides described herein can be isolated from a variety of sources, such as from human tissue types or from another source, or prepared by recombinant or synthetic methods. Examples of FOLR1 sequences include, but are not limited to NCBI reference numbers P15328, NP 001092242.1, AAX29268.1, AAX37119.1, NP 057937.1, and NP 057936.1. The human FOLR1 sequence is a follows:

(SEQ ID NO: 41)
MAQRMTTQLLLLLVWVAVVGEAQTRIAWARTELLNVCMNAKHHKEKPGPE
DKLHEQCRPWRKNACCSTNTSQEAHKDVSYLYRFNWNHCGEMAPACKRHF
IQDTCLYECSPNLGPWIQQVDQSWRKERVLNVPLCKEDCEQWWEDCRTSY
TCKSNWHKGWNWTSGFNKCAVGAACQPFHFYFPTPTVLCNEIWTHSYKVS
NYSRGSGRCIQMWFDPAQGNPNEEVARFYAAAMSGAGPWAAWPFLLSLAL
MLLWLLS.

The terms “shed antigen” and “shed FOLR1” (“sFOLR1” or “sFRα”) are used interchangeably herein. These terms refer to a FOLR1 protein that is soluble and that is not cell associated. In some embodiments it includes the extracellular domain (ECD) and the glycosylphosphatidyl inositol (GPI) linker. In one embodiment, the shed FOLR1 includes only the ECD. FOLR1 includes a signal peptide (amino acids 1-24) the FOLR1 protein chain (amino acids 25-233 or 234) and a propeptide which can be cleaved (amino acids 235 to 257). Shed FOLR can include amino acids 1 to 257, 1 to 233, 1 to 234, 25 to 233, 25 to 234 or any other fragments thereof. In some embodiments the signal sequence is cleaved. In other embodiments the ECD and the GPI portion can be embedded in a membrane (e.g., a soluble lipid raft). In one embodiment, the shed FOLR1 can include amino acids 1-233 or a fragment thereof.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

The term “anti-FOLR1 antibody” or “an antibody that binds to FOLR1” refers to an antibody that is capable of binding FOLR1 with sufficient affinity such that the antibody is useful as a diagnostic and/or therapeutic agent in targeting FOLR1 (e.g., the huMov19 (M9346A) antibody). The extent of binding of an anti-FOLR1 antibody to an unrelated, non-FOLR1 protein can be less than about 10% of the binding of the antibody to FOLR1 as measured, e.g., by a radioimmunoassay (RIA).

The term “IMGN853” (also known as mirvetuximab soravtansine) refers to the immunoconjugate described herein containing the huMov19 (M9346A) antibody, the sulfoSPDB linker, and the DM4 maytansinoid. The polypeptides of SEQ ID NOs: 56-58 comprise the variable domain of the heavy chain of huMov19 (M9346A), the variable domain light chain version 1.00, and the variable domain light chain version 1.60 of huMov19, respectively. In certain embodiments, the huMov19 (M9346A) antibody is an anti-FOLR1 antibody comprising the variable heavy chain sequence SEQ ID NO:56 and the variable light chain sequence SEQ ID NO:58 (version 1.60 of huMov19). DM4 refers to N2′-deacetyl-N2′-(4-mercapto-4-methyl-1-oxopentyl) maytansine. “SulfoSPDB” refers to the N-succinimidyl 4-(2-pyridyldithio)-2-sulfobutanoate) linker. In certain embodiments, the huMov19 (M9346A) antibody is encoded by the plasmids deposited with the American Type Culture Collection (ATCC), located at 10801 University Boulevard, Manassas, Va. 20110 on Apr. 7, 2010 under the terms of the Budapest Treaty and having ATCC deposit nos. PTA-10772 and PTA-10773 or 10774. The sequence information below provides the complementarity determining regions (CDRs), the variable heavy and light chains, and the full heavy and light chains of huMov19:

huMov19 Variable Heavy Chain
(SEQ ID NO: 56)
QVQLVQSGAEVVKPGASVKISCKASGYTFTGYFMNWVKQSPGQSLEWIGR
IHPYDGDTFYNQKFQGKATLTVDKSSNTAHMELLSLTSEDFAVYYCTRYD
GSRAMDYWGQGTTVTVSS
huMov19 Variable Light Chain version 1.00
(SEQ ID NO: 57)
DIVLTQSPLSLAVSLGQPAIISCKASQSVSFAGTSLMHWYHQKPGQQPRL
LIYRASNLEAGVPDRFSGSGSKTDFTLNISPVEAEDAATYYCQQSREYPY
TFGGGTKLEIKR
huMov19 Variable Light Chain version 1.60
(SEQ ID NO: 58)
DIVLTQSPLSLAVSLGQPAIISCKASQSVSFAGTSLMHWYHQKPGQQPRL
LIYRASNLEAGVPDRFSGSGSKTDFTLTISPVEAEDAATYYCQQSREYPY
TFGGGTKLEIKR
huMov19 Variable Light Chain CDR1
(SEQ ID NO: 59)
KASQSVSFAGTSLMH
huMov19 Variable Light Chain CDR2
(SEQ ID NO: 60)
RASNLEA
huMov19 Variable Light Chain CDR3
(SEQ ID NO: 61)
QQSREYPYT
huMov19 Variable Heavy Chain CDR1
(SEQ ID NO: 62)
GYFMN
huMov19 Variable Heavy Chain CDR2-Kabat Defined
(SEQ ID NO: 63)
RIHPYDGDTFYNQKFQG
huMov19 Variable Heavy Chain CDR2-Abm Defined
(SEQ ID NO: 64)
RIHPYDGDTF
huMov19 Variable Heavy Chain CDR3
(SEQ ID NO: 65)
YDGSRAMDY
huMov19 Heavy Chain Amino Acid Sequence
(SEQ ID NO: 66)
QVQLVQSGAEVVKPGASVKISCKASGYTFTGYFMNWVKQSPGQSLEWIGR
IHPYDGDTFYNQKFQGKATLTVDKSSNTAHMELLSLTSEDFAVYYCTRYD
GSRAMDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
huMov19 Light Chain version 1.00
Amino Acid Sequence
(SEQ ID NO: 67)
DIVLTQSPLSLAVSLGQPAIISCKASQSVSFAGTSLMHWYHQKPGQQPRL
LIYRASNLEAGVPDRFSGSGSKTDFTLNISPVEAEDAATYYCQQSREYPY
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
huMov19 Light Chain version 1.60
Amino Acid Sequence
(SEQ ID NO: 68)
DIVLTQSPLSLAVSLGQPAIISCKASQSVSFAGTSLMHWYHQKPGQQPRL
LIYRASNLEAGVPDRFSGSGSKTDFTLTISPVEAEDAATYYCQQSREYPY
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

The term “immunoconjugate” or “conjugate” as used herein refers to a compound or a derivative thereof that is linked to a cell binding agent (i.e., an anti-FOLR1 antibody or fragment thereof) and is defined by a generic formula: C-L-A, wherein C=cytotoxin, L=linker, and A=antibody or antigen-binding fragment thereof e.g., an anti-FOLR1 antibody or antibody fragment. Immunoconjugates can also be defined by the generic formula in reverse order: A-L-C.

A “linker” is any chemical moiety that is capable of linking a compound, usually a drug (such as a maytansinoid), to a cell-binding agent (such as an anti FOLR1 antibody or a fragment thereof) in a stable, covalent manner. Linkers can be susceptible to or be substantially resistant to, e.g., disulfide bond cleavage, at conditions under which the compound or the antibody remains active. Suitable linkers are well known in the art and include, for example, disulfide groups and thioether groups.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some embodiments, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.), “Kabat”); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest. (5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. (5th Ed., 1991, National Institutes of Health, Bethesda, Md.), “Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H35 |
| | | (Chothia Numbering) | |
| H2 | H50-H65 | H50-H58 | H52-H56 |
| H3 | H95-H102 | H95-H102 | H95-H102 |

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described herein.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better,” the antibody's affinity for the antigen is <0.6 nM, i.e., 0.59 nM, 0.58 nM, 0.57 nM etc. or any value less than 0.6 nM. In one embodiment, the antibody's affinity as determined by a Kd will be between about 10⁻³ to about 10⁻¹² M, between about 10⁻⁶ to about 10⁻¹¹M, between about 10⁻⁶ to about 10⁻¹⁰ M, between about 10⁻⁶ to about 10⁻⁹ M, between about 10⁻⁶ to about 10⁻⁸M, or between about 10⁻⁶ to about 10⁻⁷M.

An antibody is said to “competitively inhibit” binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

The phrase “substantially similar,” or “substantially the same,” as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristics measured by said values (e.g., Kd values). The difference between said two values is less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “increased expression” of FOLR1 refers to a sample which contains elevated levels of FOLR1 expression as compared to a reference sample, a reference FOLR1 level, or a previous FOLR1 level detected from the same subject. Thus, for example, “increased FOLR1 protein levels” in a patient sample can have FOLR1 protein levels that are higher than the FOLR1 protein levels in a non-cancerous reference sample. “Increased FOLR1 protein levels” in a patient sample can also, for example, have FOLR1 protein levels that are equal to the FOLR1 protein levels in a cancerous sample.

A “reference sample” can be used to correlate and compare the results obtained in the methods of the invention from a test sample. Reference samples can be cells (e.g., cell lines, cell pellets), bodily fluids, or tissue. The FOLR1 levels in the “reference sample” may be an absolute or relative amount, a range of amount, a minimum and/or maximum amount, a mean amount, and/or a median amount of FOLR1. A “reference sample” can also serve as a baseline of FOLR1 expression to which the test sample is compared. The “reference sample” can include a prior sample or baseline sample from the same patient, a normal reference, or a reference from a relevant patient population. Generally, FOLR1 levels are expressed as values in a standard curve. A standard curve is a quantitative method of plotting assay data to determine the concentration of FOLR1 in a sample. In one embodiment, reference sample is an antigen standard comprising purified FOLR1 or FOLR1-Fc. The methods of detection disclosed herein may involve a comparison between expression levels of FOLR1 in a test sample and a “reference value” or “referece level.” In some embodiments, the reference value is the expression level of the FOLR1 in a reference sample. A reference value may be a predetermined value and may also be determined from reference samples (e.g., control biological samples) tested in parallel with the test samples. A reference value can be a single cut-off value, such as a median or mean or a range of values, such as a confidence interval. Reference values can be established for various subgroups of individuals, such as individuals predisposed to cancer, individuals having early or late stage cancer, male and/or female individuals, or individuals undergoing cancer therapy. Examples of normal reference samples or values and positive reference samples or values are described herein.

A “sample” or “biological sample” of the present invention is of biological origin, in specific embodiments, such as from eukaryotic organisms. In preferred embodiments, the sample is a human sample, but animal samples may also be used in the practice of the invention. Non-limiting sources of a sample for use in the present invention include solid tissue, biopsy aspirates, ascites, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example. The present invention is particularly useful for cancer samples which generally comprise bodily fluids such as ascites, where the amount of available material is small. The method can be used to examine an aspect of expression of FOLR1 or a state of a sample, including, but not limited to, comparing different types of cells or tissues, comparing different developmental stages, and detecting or determining the presence and/or type of disease or abnormality.

As used herein, the term “capture reagent” refers to a reagent capable of binding and capturing a target molecule in a sample such that under suitable condition, the capture reagent-target molecule complex can be separated from the rest of the sample. The term “immunocapture reagent” refers to an immunological reagent that is capable of binding and capturing a target molecule in a sample such that under suitable conditions, the capture reagent-target molecule complex can be separated from the rest of the sample. In one embodiment, the immunocapture reagent is an antibody or antigen-binding fragment. In one embodiment, the capture reagent or immunocoapture reagent is immobilized. In one embodiment, the capture reagent or immunocapture reagent is immobilized on a solid support.

As used herein, the term “detectable antibody” refers to an antibody that is capable of being detected either directly through a label amplified by a detection means, or indirectly through, e.g., another antibody that is labeled. For direct labeling, the antibody is typically conjugated to a moiety that is detectable by some means. In one embodiment, the detectable antibody is a biotinylated antibody.

The word “label” when used herein refers to a detectable compound or composition which is conjugated directly or indirectly to the antibody so as to generate a “labeled” antibody. The label can be detectable by itself (e.g., radioisotope labels or fluorescent labels) or, in the case of an enzymatic label, can catalyze chemical alteration of a substrate compound or composition which is detectable.

By “correlate” or “correlating” is meant comparing, in any way, the performance and/or results of a first analysis with the performance and/or results of a second analysis. For example, one may use the results of a first analysis in carrying out the second analysis and/or one may use the results of a first analysis to determine whether a second analysis should be performed and/or one may compare the results of a first analysis with the results of a second analysis.

The terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers. The cancer can be a cancer that expresses FOLR1.

“Tumor” and “neoplasm” refer to any mass of tissue that result from excessive cell growth or proliferation, either benign (noncancerous) or malignant (cancerous) including pre-cancerous lesions.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical formulation” refers to a preparation which is in such form as to permit the biological activity of the active ingredient to be effective, and which contains no additional components which are unacceptably toxic to a subject to which the formulation would be administered. Such formulation can be sterile.

An “effective amount” of an antibody as disclosed herein is an amount sufficient to carry out a specifically stated purpose. An “effective amount” can be determined empirically and in a routine manner, in relation to the stated purpose.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains. In some embodiments, a polypeptide, peptide, or protein is non-naturally occurring. In some embodiments, a polypeptide, peptide, or protein is purified from other naturally occurring components. In some embodiments, the polypeptide, peptide, or protein is recombinantly produced.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482 489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In certain embodiments, identity exists over a region of the sequences that is at least about 10, about 20, about 40-60 residues in length or any integral value therebetween, or over a longer region than 60-80 residues, at least about 90-100 residues, or the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the invention do not abrogate the binding of the polypeptide or antibody containing the amino acid sequence, to the antigen(s), i.e., the FOLR1 to which the polypeptide or antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al. Protein Eng. 12(10):879-884 (1999); and Burks et al. Proc. Natl. Acad. Sci. USA 94:412-417 (1997)).

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. FOLR1-Binding Agents

Agents that specifically bind human FOLR1 are useful in performing the methods of the invention. Such agents are referred to herein as “FOLR1-binding agents.” FOLR1-binding agents useful in performing the methods of the present invention are described in, e.g., WO 2014/036495 and WO 2015/031815, which are incorporated herein by reference in their entireties.

The FOLR1-binding agents include agents that comprise the heavy and light chain CDR sequences of muFR1-9, muFR1-13, muFR1-53, muFR1-62, muFR1-64, and FOLR1-2.1 (also referred to as “IMGN 353.2-1,” “353.2-1,” “2.1,” or “muFRIHC2-1”). The CDR sequences muFR1-9, muFR1-13, muFR1-53, muFR1-62, FOLR1-2.1 are described in Tables 1 and 2 below.

TABLE 1
Variable heavy chain CDR amino acid sequences

| Antibody | VH-CDR1 | VH-CDR2 | VH-CDR3 |
|---|---|---|---|
| muFR1-9 | SFGMH | YISSGSSTFYYADTVKG | ELTGTFAY |
| | (SEQ ID NO: 1) | (SEQ ID NO: 2) | (SEQ ID NO: 3) |
| muFR1-13 | RYSVH | MIWSGGNTDYNSVFKS | FDGKVSWFAY |
| | (SEQ ID NO: 4) | (SEQ ID NO: 5) | (SEQ ID NO: 6) |
| muFR1-53 | DYDIS | EIYPGSGRTYYNERFKG | SYYYGTNSPFAY |
| | (SEQ ID NO: 7) | (SEQ ID NO: 8) | (SEQ ID NO: 9) |
| muFR1-62 | TYTMH | YINPTSGYNNYNQKFKE | GGAYGRRPVDY |
| | (SEQ ID NO: 10) | (SEQ ID NO: 11) | (SEQ ID NO: 12) |
| muFRIHC2-1 | NSYIH | WIYPESLNTQYNEKFKA | RGIYYYSPYALDH |
| (“2.1”) | (SEQ ID NO: 46) | (SEQ ID NO: 47) | (SEQ ID NO: 48) |

TABLE 2
Variable light chain CDR amino acid sequences

| Antibody | VL-CDR1 | VL-CDR2 | VL-CDR3 |
|---|---|---|---|
| muFR1-9 | RASQSINNNLH | YASQSIS | QQSNSWPQVT |
| | (SEQ ID NO: 13) | (SEQ ID NO: 14) | (SEQ ID NO: 15) |
| muFR1-13 | KASQSVSNDVL | YAYNRYS | QQDHSSPFT |
| | (SEQ ID NO: 16) | (SEQ ID NO: 17) | (SEQ ID NO: 18) |
| muFR1-53 | RASQDISNYLH | YTSRLQS | QQGNSLPPT |
| | (SEQ ID NO: 19) | (SEQ ID NO: 20) | (SEQ ID NO: 21) |
| muFR1-62 | KASQNVGTNVA | SASSRYS | HQYNSYPYT |
| | (SEQ ID NO: 22) | (SEQ ID NO: 23) | (SEQ ID NO: 24) |
| muFRIHC2-1 | KSSKSLLNSDGFTYLD | LVSNHFS | FQSNYLPLT |
| (“2.1”) | (SEQ ID NO: 49) | (SEQ ID NO: 50) | (SEQ ID NO: 51) |

The FOLR1 binding molecules can be antibodies or antigen-binding fragments that specifically bind to FOLR1 that comprise the CDRs of muFR1-9, muFR1-13, muFR1-53, muFR1-62, muFR1-64, or FOLR1-2.1 (also referred to as “IMGN 353.2-1,” “353.2-1,” “2.1,” or “muFRIHC2-1”) with up to four (i.e., 0, 1, 2, 3, or 4) conservative amino acid substitutions per CDR.

Polypeptides can comprise one of the individual variable light chains or variable heavy chains described herein. Antibodies and polypeptides can also comprise both a variable light chain and a variable heavy chain. The variable light chain and variable heavy chain sequences of murine muFR1-9, muFR1-13, muFR1-53, muFR1-62, and FOLR1-2.1 (also referred to as “IMGN 353.2-1,” “353.2-1,” “2.1,” or “muFRIHC2-1”) antibodies are provided in Tables 3 and 4 below.

TABLE 3
Variable heavy chain amino acid sequences

| Antibody | VH Amino Acid Sequence (SEQ ID NO) |
|---|---|
| muFR1-9HCvar | QVQLVESGGGLVQPGGSRKLSCAASGFTFSSFGMH |
| | WVRQAPEKGLEWVAYISSGSSTFYYADTVKGRFTI |
| | SRDNPKNTLFLQMTSLRSEDTAMYYCAKELTGTFA |
| | YWGQGTLVTVSA (SEQ ID NO: 25) |
| muFR1-13HCvar | QVQLKESGPDLVAPSQSLSITCTVSGFSLSRYSVH |
| | WIRQPPGKGLEWLGMIWSGGNTDYNSVFKSRLNIT |
| | KDNSKSQVFLKMNSLQTDDTAIYYCATFDGKVSWF |
| | AYWGQGTLVTVSA (SEQ ID NO: 26) |
| muFR1-53HC | QVQLQQSGPELVRPGASVKMSCKASGYKFTDYDIS |
| | WVLQRTGQGLEWIGEIYPGSGRTYYNERFKGKATL |
| | TADKSSNTVYMQLSSLTSEDSAVYFCASSYYYGTN |
| | SPFAYWGQGTTLTVSS (SEQ ID NO: 27) |
| muFR1-62HC | QVQLQQSGAELARPGASVKMSCKASGYTFTTYTMH |
| | WVKQRPGQGLEWIAYINPTSGYNNYNQKFKEKATL |
| | TADKSSSTAYMQLTSLTSEDSAVYYCASGGAYGRR |
| | PVDYWGQGTSVTVSS (SEQ ID NO: 28) |
| muFRIHC2-1 | QVQLQQSGPELVKPGASVRISCKASGYTFTNSYIH |
| (“2.1”) | WVKKRPGQGLEWIGWIYPESLNTQYNEKFKAKATL |
| | TADKSSSTSYMQLSSLTSEDSAVYFCARRGIYYYS |
| | PYALDHWGQGASVTVSS (SEQ ID NO: 52) |

TABLE 4
Variable light chain amino acid sequences

| Antibody | VL Amino Acid Sequence (SEQ ID NO) |
|---|---|
| muFR1-9Lcvar | DIVLTQSPATLSVTPGDSVSLSCRASQSINNNLHW |
| | YQQKSHESPRLLIKYASQSISGIPSRFSGSGSGTD |
| | FTLSINSVETEDFGMYFCQQSNSWPQVTFGAGTKL |
| | ELKR (SEQ ID NO: 29) |
| muFR1-13LCvar | SIVMTQTPKFLLVSTGDRFTITCKASQSVSNDVLW |
| | YQQKPGQSPKLLIYYAYNRYSGVPDRFTGSGYGTD |
| | FTFTITTVQSEDLAVYFCQQDHSSPFTFGSGTKLE |
| | IKR (SEQ ID NO: 30) |
| muFR1-53LC | DIQMTQTTSSLSASLGDRVTISCRASQDISNYLHW |
| | YQRKPDGTVKLLVYYTSRLQSGVPSRFSGSGSGTD |
| | YSLTISNLEQEDIATYFCQQGNSLPPTFGSGTKLE |
| | IKR (SEQ ID NO: 31) |
| muFR1-62LC | DIVMTQSQKFMSISVGDRVSVTCKASQNVGTNVAW |
| | YQQKPGQSPKTLIYSASSRYSGVPDRFTGSGSGTD |
| | FTLTISNVQSEDLADYFCHQYNSYPYTFGGGTKLE |
| | IKR (SEQ ID NO: 32) |
| muFRIHC2-1 | SDVVLTQTPLSLPVNIGDQASISCKSSKSLLNSDG |
| (“2.1”) | FTYLDWYLQKPGQSPQLLIYLVSNHFSGVPDRFSG |
| | SGSGTDFTLKISRVEAEDLGVYYCFQSNYLPLTFG |
| | GGTKLEIKR (SEQ ID NO: 53) |

Also provided are polypeptides that comprise: (a) a polypeptide having at least about 90% sequence identity to SEQ ID NOs:25-28; and/or (b) a polypeptide having at least about 90% sequence identity to SEQ ID NOs:29-32. In certain embodiments, the polypeptide comprises a polypeptide having at least about 95%, at least about 96%, at least about 97%, at least about 98%, or at least about 99% sequence identity to SEQ ID NOs:25-32. Thus, in certain embodiments, the polypeptide comprises (a) a polypeptide having at least about 95% sequence identity to SEQ ID NOs:25-28, and/or (b) a polypeptide having at least about 95% sequence identity to SEQ ID NOs:29-32. In certain embodiments, the polypeptide comprises (a) a polypeptide having the amino acid sequence of SEQ ID NOs:25-28; and/or (b) a polypeptide having the amino acid sequence of SEQ ID NOs:29-32. In certain embodiments, the polypeptide is an antibody and/or the polypeptide specifically binds FOLR1. In certain embodiments, the polypeptide is a murine, chimeric, or humanized antibody that specifically binds FOLR1. In certain embodiments, the polypeptide having a certain percentage of sequence identity to SEQ ID NOs:25-32 differs from SEQ ID NOs:25-32 by conservative amino acid substitutions only.

Polypeptides can comprise one of the individual light chains or heavy chains described herein. Antibodies and polypeptides can also comprise both a light chain and a heavy chain. The light chain and variable chain sequences of murine muFR1-9, muFR1-13, muFR1-53, muFR1-62, and FOLR1-2.1 (also referred to as “IMGN 353.2-1,” “353.2-1,” “2.1,” or “muFRIHC2-1”) antibodies are provided in Tables 5 and 6 below. In certain embodiments, the anti-FOLR1 antibody is the antibody produced by the hybridoma deposited with the ATCC on Apr. 16, 2013 and having ATCC deposit no. PTA-120197 (“FOLR1-2.1,” also referred to as “IMGN 353.2-1,” “353.2-1,” “2.1,” or “muFRIHC2-1”).

TABLE 5
Full-length heavy chain amino acid sequences

| | Full-Length Heavy Chain |
|---|---|
| Antibody | Amino Acid Sequence (SEQ ID NO) |
| muFR1-9HC | QVQLVESGGGLVQPGGSRKLSCAASGFTFSSFGMHWVRQ |
| | APEKGLEWVAYISSGSSTFYYADTVKGRFTISRDNPKNT |
| | TGTFAYWGQGTLVTVSAAKTTPPSVYPLAPGSAAQTNSM |
| | VTLFLQMTSLRSEDTAMYYCAKELLGCLVKGYFPEPVTV |
| | TWNSGSLSSGVHTFPAVLESDLYTLSSSVTVPSSMRPSE |
| | TVTCNVAHPASSTKVDKKIVPRDCGCKPCICTVPEVSSV |
| | FIFPPKPKDVLTITLTPKVTCVVVDISKDDPEVQFSWFV |
| | DDVEVHTAQTQPREEQFNSTFRSVSELPIMHQDWLNGKE |
| | FKCRVNSAAFPAPIEKTISKTKGRPKAPQVYTIPPPKEQ |
| | MAKDKVSLTCMITDFFPEDITVEWQWNGQPAENYKNTQP |
| | IMNTNGSYFVYSKLNVQKSNWEAGNTFTCSVLHEGLHNH |
| | HTEKSLSHSPGK (SEQ ID NO: 33) |
| muFR1-13HC | QVQLKESGPDLVAPSQSLSITCTVSGFSLSRYSVHWIRQ |
| | PPGKGLEWLGMIWSGGNTDYNSVFKSRLNITKDNSKSQV |
| | FLKMNSLQTDDTAIYYCATFDGKVSWFAYWGQGTLVTVS |
| | AAKTTPPSVYPLAPGCGDTTGSSVTLGCLVKGYFPESVT |
| | VTWNSGSLSSSVHTFPALLQSGLYTMSSSVTVPSSTWPS |
| | QTVTCSVAHPASSTTVDKKLEPSGPISTINPCPPCKECH |
| | KCPAPNLEGGPSVFIFPPNIKDVLMISLTPKVTCVVVDV |
| | SEDDPDVQISWFVNNVEVHTAQTQTHREDYNSTIRVVST |
| | LPIQHQDWMSGKEFKCKVNNKDLPSPIERTISKIKGLVR |
| | APQVYILPPPAEQLSRKDVSLTCLVVGFNPGDISVEWTS |
| | NGHTEENYKDTAPVLDSDGSYFIYSKLNMKTSKWEKTDS |
| | FSCNVRHEGLKNYYLKKTISRSPGK (SEQ ID |
| | NO: 34) |
| muFR1-53HC | QVQLQQSGPELVRPGASVKMSCKASGYKFTDYDISWVLQ |
| | RTGQGLEWIGEIYPGSGRTYYNERFKGKATLTADKSSNT |
| | VYMQLSSLTSEDSAVYFCASSYYYGTNSPFAYWGQGTTL |
| | TVSSAKTTPPSVYPLAPGSAAQTNSMVTLGCLVKGYFPE |
| | PVTVTWNSGSLSSGVHTFPAVLESDLYTLSSSVTVPSSM |
| | RPSETVTCNVAHPASSTKVDKKIVPRDCGCKPCICTVPE |
| | VSSVFIFPPKPKDVLTITLTPKVTCVVVDISKDDPEVQF |
| | SWFVDDVEVHTAQTQPREEQFNSTFRSVSELPIMHQDWL |
| | NGKEFKCRVNSAAFPAPIEKTISKTKGRPKAPQVYTIPP |
| | PKEQMAKDKVSLTCMITDFFPEDITVEWQWNGQPAENYK |
| | NTQPIMNTNGSYFVYSKLNVQKSNWEAGNTFTCSVLHEG |
| | LHNHHTEKSLSHSPGK (SEQ ID NO: 35) |
| muFR1-62HC | QVQLQQSGAELARPGASVKMSCKASGYTFTTYTMHWVKQ |
| | RPGQGLEWIAYINPTSGYNNYNQKFKEKATLTADKSSST |
| | AYMQLTSLTSEDSAVYYCASGGAYGRRPVDYWGQGTSVT |
| | VSSAKTTPPSVYPLAPGSAAQTNSMVTLGCLVKGYFPEP |
| | VTVTWNSGSLSSGVHTFPAVLESDLYTLSSSVTVPSSMR |
| | PSETVTCNVAHPASSTKVDKKIVPRDCGCKPCICTVPEV |
| | SSVFIFPPKPKDVLTITLTPKVTCVVVDISKDDPEVQFS |
| | WFVDDVEVHTAQTQPREEQFNSTFRSVSELPIMHQDWLN |
| | GKEFKCRVNSAAFPAPIEKTISKTKGRPKAPQVYTIPPP |
| | KEQMAKDKVSLTCMITDFFPEDITVEWQWNGQPAENYKN |
| | TQPIMNTNGSYFVYSKLNVQKSNWEAGNTFTCSVLHEGL |
| | HNHHTEKSLSHSPGK (SEQ ID NO: 36) |
| muFRIHC2-1 | QVQLQQSGPELVKPGASVRISCKASGYTFTNSYIHWVKK |
| (“2.1”) | RPGQGLEWIGWIYPESLNTQYNEKFKAKATLTADKSSST |
| | SYMQLSSLTSEDSAVYFCARRGIYYYSPYALDHWGQGAS |
| | VTVSSAKTTPPSVYPLAPGSAAQTNSMVTLGCLVKGYFP |
| | EPVTVTWNSGSLSSGVHTFPAVLESDLYTLSSSVTVPSS |
| | MRPSETVTCNVAHPASSTKVDKKIVPRDCGCKPCICTVP |
| | EVSSVFIFPPKPKDVLTITLTPKVTCVVVDISKDDPEVQ |
| | FSWFVDDVEVHTAQTQPREEQFNSTFRSVSELPIMHQDW |
| | LNGKEFKCRVNSAAFPAPIEKTISKTKGRPKAPQVYTIP |
| | PPKEQMAKDKVSLTCMITDFFPEDITVEWQWNGQPAENY |
| | KNTQPIMNTNGSYFVYSKLNVQKSNWEAGNTFTCSVLHE |
| | GLHNHHTEKSLSHSPGK (SEQ ID NO: 54) |

TABLE 6
Full-length light chain amino acid sequences

| | Full-length Light Chain |
|---|---|
| Antibody | Amino Acid Sequence (SEQ ID NO) |
| muFR1-9LC | DIVLTQSPATLSVTPGDSVSLSCRASQSINNNLHWYQQK |
| | SHESPRLLIKYASQSISGIPSRFSGSGSGTDFTLSINSV |
| | ETEDFGMYFCQQSNSWPQVTFGAGTKLELKRADAAPTVS |
| | IFPPSSEQLTSGGASVVCFLNNFYPKDINVKWKIDGSER |
| | QNGVLNSWTDQDSKDSTYSMSSTLTLTKDEYERHNSYTC |
| | EATHKTSTSPIVKSFNRNEC (SEQ ID NO: 37) |
| muFR1-13LC | SIVMTQTPKFLLVSTGDRFTITCKASQSVSNDVLWYQQK |
| | PGQSPKLLIYYAYNRYSGVPDRFTGSGYGTDFTFTITTV |
| | QSEDLAVYFCQQDHSSPFTFGSGTKLEIKRADAAPTVSI |
| | FPPSSEQLTSGGASVVCFLNNFYPKDINVKWKIDGSERQ |
| | NGVLNSWTDQDSKDSTYSMSSTLTLTKDEYERHNSYTCE |
| | ATHKTSTSPIVKSFNRNEC (SEQ ID NO: 38) |
| muFR1-53LC | DIQMTQTTSSLSASLGDRVTISCRASQDISNYLHWYQRK |
| | PDGTVKLLVYYTSRLQSGVPSRFSGSGSGTDYSLTISNL |
| | EQEDIATYFCQQGNSLPPTFGSGTKLEIKRADAAPTVSI |
| | FPPSSEQLTSGGASVVCFLNNFYPKDINVKWKIDGSERQ |
| | NGVLNSWTDQDSKDSTYSMSSTLTLTKDEYERHNSYTCE |
| | ATHKTSTSPIVKSFNRNEC (SEQ ID NO: 39) |
| muFR1-62LC | DIVMTQSQKFMSISVGDRVSVTCKASQNVGTNVAWYQQK |
| | PGQSPKTLIYSASSRYSGVPDRFTGSGSGTDFTLTISNV |
| | QSEDLADYFCHQYNSYPYTFGGGTKLEIKRADAAPTVSI |
| | FPPSSEQLTSGGASVVCFLNNFYPKDINVKWKIDGSERQ |
| | NGVLNSWTDQDSKDSTYSMSSTLTLTKDEYERHNSYTCE |
| | ATHKTSTSPIVKSFNRNEC (SEQ ID NO: 40) |
| muFRIHC2-1 | SDVVLTQTPLSLPVNIGDQASISCKSSKSLLNSDGFTYL |
| (“2.1”) | DWYLQKPGQSPQLLIYLVSNHFSGVPDRFSGSGSGTDFT |
| | LKISRVEAEDLGVYYCFQSNYLPLTFGGGTKLEIKRADA |
| | APTVSIFPPSSEQLTSGGASVVCFLNNFYPKDINVKWKI |
| | DGSERQNGVLNSWTDQDSKDSTYSMSSTLTLTKDEYERH |
| | NSYTCEATHKTSTSPIVKSFNRNEC (SEQ ID |
| | NO: 55) |

Also provided are polypeptides that comprise: (a) a polypeptide having at least about 90% sequence identity to SEQ ID NOs:33-36; and/or (b) a polypeptide having at least about 90% sequence identity to SEQ ID NOs:37-40. In certain embodiments, the polypeptide comprises a polypeptide having at least about 95%, at least about 96%, at least about 97%, at least about 98%, or at least about 99% sequence identity to SEQ ID NOs:33-40. Thus, in certain embodiments, the polypeptide comprises (a) a polypeptide having at least about 95% sequence identity to SEQ ID NOs:33-36, and/or (b) a polypeptide having at least about 95% sequence identity to SEQ ID NOs:37-40. In certain embodiments, the polypeptide comprises (a) a polypeptide having the amino acid sequence of SEQ ID NOs:33-36; and/or (b) a polypeptide having the amino acid sequence of SEQ ID NOs:37-40. In certain embodiments, the polypeptide is an antibody and/or the polypeptide specifically binds FOLR1. In certain embodiments, the polypeptide is a murine, chimeric, or humanized antibody that specifically binds FOLR1. In certain embodiments, the polypeptide having a certain percentage of sequence identity to SEQ ID NOs:33-40 differs from SEQ ID NOs:33-40 by conservative amino acid substitutions only.

In certain embodiments, the polypeptide is not competitively inhibited by binding of huMov19 to FOLR1. In certain embodiments, the polypeptide is not competitively inhibited by binding of IMGN853 to FOLR1. In certain embodiments, the polypeptide is not competitively inhibited by binding of huMov19 to FOLR1. In certain embodiments, the polypeptide is not competitively inhibited by binding of an antibody or antigen-binding fragment to FOLR1, wherein said antibody or antigen-binding fragment comprises: a variable light chain (VL) complementarity determining region (CDR)-1 of SEQ ID NO: 59; a VL CDR-2 of SEQ ID NO: 60; a VL CDR-3 of SEQ ID NO: 61; a variable heavy chain (VH) CDR-1 of SEQ ID NO: 62; a VH CDR-2 of SEQ ID NO: 64; and a VH CDR-3 of SEQ ID NO: 65. In certain embodiments, the polypeptide is not competitively inhibited by binding of an antibody or antigen-binding fragment to FOLR1, wherein said antibody or antigen-binding fragment comprises a variable heavy chain (VH) having the sequence of SEQ ID NO: 56 and a variable light chain (VL) having the sequence of SEQ ID NO: 57 or SEQ ID NO: 58. In certain embodiments, the polypeptide is not competitively inhibited by binding of an antibody or antigen-binding fragment to FOLR1, wherein said antibody or antigen-binding fragment comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774. In certain embodiments, the polypeptide is not inhibited by binding of folic acid to FOLR1.

The affinity or avidity of an antibody for an antigen can be determined experimentally using any suitable method well known in the art, e.g., cytometry (including flow cytometry), enzyme-linked immunoabsorbent assay (ELISA), or radioimmunoassay (MA), or kinetics (e.g., surface plasmon resonance spectroscopy (BIACORE™) analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., KD or Kd, Kₒₙ, K_{off}) are made with standardized solutions of antibody and antigen, and a standardized buffer, as known in the art and such as the buffer described herein.

In one aspect, binding assays can be performed using cytometry (e.g., flow cytometry) on cells expressing the FOLR1 antigen on the surface. For example, FOLR1-positve cells such as SKOV3 were incubated with varying concentrations of anti-FOLR1 antibodies using 1×10⁵ cells per sample in 100 μL FACS buffer (RPMI-1640 medium supplemented with 2% normal goat serum). Then, the cells were pelleted, washed, and incubated for 1 h with 100 μL of FITC-conjugated goat-anti-mouse or goat-anti-human IgG-antibody (such as is obtainable from, for example Jackson Laboratory, 6 μg/mL in FACS buffer). The cells were pelleted again, washed with FACS buffer and resuspended in 200 μL of PBS containing 1% formaldehyde. Samples were acquired, for example, using a FACSCalibur flow cytometer with the HTS multiwell sampler and analyzed using CellQuest Pro (all from BD Biosciences, San Diego, US). For each sample the mean fluorescence intensity for FL1 (MFI) was exported and plotted against the antibody concentration in a semi-log plot to generate a binding curve. A sigmoidal dose-response curve is fitted for binding curves and EC50 values are calculated using programs such as GraphPad Prism v4 with default parameters (GraphPad software, San Diego, Calif.). EC50 values can be used as a measure for the apparent dissociation constant “Kd” or “KD” for each antibody.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g., radioimmunoassay (RIA); enzyme-linked immunosorbent assay (ELISA)) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies.

Alternatively monoclonal antibodies can also be made using recombinant DNA methods as described in U.S. Pat. No. 4,816,567. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the monoclonal antibody against the human FOLR1 is a humanized antibody. In certain embodiments, such antibodies are used therapeutically to reduce antigenicity and HAMA (human anti-mouse antibody) responses when administered to a human subject.

Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing FOLR1 binding. Accordingly, part or all of the non-human or human CDR sequences are maintained while the non-human sequences of the variable and constant regions can be replaced with human or other amino acids.

Antibodies can also optionally be humanized, resurfaced, engineered or human antibodies engineered with retention of high affinity for the antigen FOLR1 and other favorable biological properties. To achieve this goal, humanized (or human) or engineered anti-FOLR1 antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as FOLR1. In this way, framework (FR) residues can be selected and combined from the consensus and import sequences so that the desired antibody characteristic, such as increased affinity for the target antigen(s), is achieved.

Humanization, resurfacing or engineering of antibodies disclosed herein can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Roguska et al., Proc. Natl. Acad. Sci., USA, 91(3):969-973 (1994), Roguska et al., Protein Eng. 9(10):895-904 (1996), U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; PCT/: US98/16280; US96/18978; US91/09630; US91/05939; US94/01234; GB89/01334; GB91/01134; GB92/01755; WO90/14443; WO90/14424; WO90/14430; EP 229246; 7,557,189; 7,538,195; and 7,342,110, each of which is entirely incorporated herein by reference, including the references cited therein.

The agents that specifically bind to FOLR1 disclosed herein also encompass antibody fragments. Various techniques are known for the production of antibody fragments. Traditionally, these fragments are derived via proteolytic digestion of intact antibodies (for example Morimoto et al., 1993, Journal of Biochemical and Biophysical Methods 24:107-117; Brennan et al., 1985, Science, 229:81). In certain embodiments, antibody fragments are produced recombinantly. Fab, Fv, and scFv antibody fragments can all be expressed in and secreted from E. coli or other host cells, thus allowing the production of large amounts of these fragments. Such antibody fragments can also be isolated from the antibody phage libraries discussed above. The antibody fragment can also be linear antibodies as described in U.S. Pat. No. 5,641,870, for example, and can be monospecific or bispecific. Other techniques for the production of antibody fragments will be apparent to the skilled practitioner.

For the purposes of the present invention, it should be appreciated that modified antibodies can comprise any type of variable region that provides for the association of the antibody with the polypeptides of a human FOLR1. In this regard, the variable region can comprise or be derived from any type of mammal that can be induced to mount a humoral response and generate immunoglobulins against the desired tumor associated antigen. As such, the variable region of the modified antibodies can be, for example, of human, murine, non-human primate (e.g., cynomolgus monkeys, macaques, etc.) or lupine origin. In some embodiments both the variable and constant regions of the modified immunoglobulins are human. In other embodiments the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It may not be necessary to replace all of the CDRs with the complete CDRs from the donor variable region to transfer the antigen-binding capacity of one variable domain to another. Rather, it may only be necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that the modified antibodies disclosed herein will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. That is, the modified antibodies disclosed herein can comprise alterations or modifications to one or more of the three heavy chain constant domains (CHL CH2 or CH3) and/or to the light chain constant domain (CL). In some embodiments, modified constant regions wherein one or more domains are partially or entirely deleted are contemplated. In some embodiments, the modified antibodies will comprise domain deleted constructs or variants wherein the entire CH2 domain has been removed (ΔCH2 constructs). In some embodiments, the omitted constant region domain will be replaced by a short amino acid spacer (e.g., 10 residues) that provides some of the molecular flexibility typically imparted by the absent constant region.

It will be noted that in certain embodiments, the modified antibodies can be engineered to fuse the CH3 domain directly to the hinge region of the respective modified antibodies. In other constructs it may be desirable to provide a peptide spacer between the hinge region and the modified CH2 and/or CH3 domains. For example, compatible constructs could be expressed wherein the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic, or even omitted altogether, so as to maintain the desired biochemical qualities of the modified antibodies.

Besides the deletion of whole constant region domains, it will be appreciated that the antibodies disclosed herein can be provided by the partial deletion or substitution of a few or even a single amino acid. For example, the mutation of a single amino acid in selected areas of the CH2 domain may be enough to substantially reduce Fc binding and thereby increase tumor localization. Similarly, it may be desirable to simply delete that part of one or more constant region domains that control the effector function (e.g., complement C1Q binding) to be modulated. Such partial deletions of the constant regions can improve selected characteristics of the antibody (serum half-life) while leaving other desirable functions associated with the subject constant region domain intact. Moreover, as alluded to above, the constant regions of the disclosed antibodies can be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. Certain embodiments can comprise the addition of one or more amino acids to the constant region to enhance desirable characteristics such as decreasing or increasing effector function or provide for more cytotoxin or carbohydrate attachment. In such embodiments it can be desirable to insert or replicate specific sequences derived from selected constant region domains.

The antibodies disclosed herein also include variants and equivalents which are substantially homologous to the chimeric, humanized and human antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

The polypeptides of the FOLR1-binding agents disclosed herein can be recombinant polypeptides, natural polypeptides, or synthetic polypeptides comprising an antibody, or fragment thereof, against a human FOLR1. It will be recognized in the art that some amino acid sequences of the invention can be varied without significant effect of the structure or function of the protein. Thus, the FOLR1-binding agents disclosed herein also include variations of the polypeptides which show substantial activity or which include regions of an antibody, or fragment thereof, against a human folate receptor protein. Such mutants include deletions, insertions, inversions, repeats, and type substitutions.

The polypeptides and analogs can be further modified to contain additional chemical moieties not normally part of the protein. Those derivatized moieties can improve the solubility, the biological half life or absorption of the protein. The moieties can also reduce or eliminate any desirable side effects of the proteins and the like. An overview for those moieties can be found in REMINGTON'S PHARMACEUTICAL SCIENCES, 20th ed., Mack Publishing Co., Easton, Pa. (2000).

### III. Methods of Detection

The methods of the present invention provide a multi-step approach to detect human FOLR1 in a sample. In particular, an initial immunocapture step is performed to enrich for FOLR1 in a sample, followed by digestion of the FOLR1 into peptides and analysis by liquid chromatography-mass spectrometry (LC/MS). Analysis of the peptides by LC/MS further allows the level of FOLR1 present in a sample to be quantitatively determined, including, for example, the level of FOLR1 in a sample from a patient with a FOLR1-expressing cancer. In some embodiments, the method of detecting human folate receptor 1 (FOLR1) in a sample comprises: (a) capturing said folate receptor 1 (FOLR1) with an immunocapture reagent bound to a solid support; (b) eluting FOLR1 from the solid support; (c) digesting the eluted FOLR1; and (d) performing liquid chromatography-mass spectrometry (LC/MS) analysis on the digested FOLR1, wherein said FOLR1 is detected by monitoring the chromatographic separation and mass spectrometric response of at least one signature FOLR1 peptide. Compared to other known methods for the detection of FOLR1, the methods of the present invention provide the advantage of enhanced sensitivity and selectivity.

In some embodiments, the sample containing FOLR1 comprises a bodily fluid. In further embodiments, the bodily fluid may be plasma, serum, or ascites fluid. In a particular embodiment, the sample is plasma. In some embodiments, the sample comprises a peripheral blood sample. In some embodiments, the sample is obtained from a patient having cancer. In further embodiments, the cancer is selected from the group consisting of: ovarian cancer, lung cancer, colorectal cancer, pancreatic cancer, liver cancer, breast cancer, brain cancer, kidney cancer, prostate cancer, gastrointestinal cancer, melanoma, cervical cancer, bladder cancer, glioblastoma, endometrial cancer, cancer of the peritoneum, and head and neck cancer. In certain embodiments, the cancer is ovarian cancer. In certain embodiments, the cancer is endometrial cancer. In certain embodiments, the cancer is lung cancer. In certain embodiments, the lung cancer is non small cell lung cancer. In certain embodiments, the non-small cell lung cancer is adenocarcinoma of the lung.

a. Immunocapture

An initial immunocapture step is performed on the sample using an immunocapture reagent bound to a solid support. The immunocapture reagent may be any immunological reagent which binds to FOLR1, including, for example, an antibody which binds to FOLR1 or an antigen-binding fragment thereof. When an antibody or antigen-binding fragment is used as an immunocapture reagent, the binding of the antibody or antigen-binding fragment to FOLR1 may not be competitively inhibited by binding of an antibody-based active agent, such as IMGN853, to FOLR1 in the sample. Moreover, when an antibody or antigen-binding fragment is used as an immunocapture reagent, the binding of the antibody or antigen-binding fragment to FOLR1 may not be inhibited by folic acid present in the sample.

In some embodiments, the immunocapture reagent is the antibody muFR1-9. In some embodiments, the immunocapture reagent is the antibody muFR1-13. In other embodiments, the immunocapture reagent is muFR1-53. In other embodiments, the immunocapture reagent is the antibody muFR1-62.

In some embodiments, the immunocapture reagent is an antibody or antigen-binding fragment which comprises a variable heavy chain (VH) complementarity determining region (CDR)-1 of SEQ ID NO: 1; a VH CDR-2 of SEQ ID NO: 2; a VH CDR-3 of SEQ ID NO: 3; a variable light chain (VL) complementarity determining region (CDR)-1 of SEQ ID NO: 13; a VL CDR-2 of SEQ ID NO: 14; and a VL CDR-3 of SEQ ID NO: 15. In some embodiments, the immunocapture reagent is an antibody or antigen-binding fragment which comprises a VH CDR-1 of SEQ ID NO: 4; a VH CDR-2 of SEQ ID NO: 5; a VH CDR-3 of SEQ ID NO: 6; a VL CDR-1 of SEQ ID NO: 16; a VL CDR-2 of SEQ ID NO: 17; and a VL CDR-3 of SEQ ID NO: 18. In some embodiments, the immunocapture reagent is an antibody or antigen-binding fragment which comprises a VH CDR-1 of SEQ ID NO: 7; a VH CDR-2 of SEQ ID NO: 8; a VH CDR-3 of SEQ ID NO: 9; a VL CDR-1 of SEQ ID NO: 19; a VL CDR-2 of SEQ ID NO: 20; and a VL CDR-3 of SEQ ID NO: 21. In some embodiments, the immunocapture reagent is an antibody or antigen-binding fragment which comprises a VH CDR-1 of SEQ ID NO: 10; a VH CDR-2 of SEQ ID NO: 11; a VH CDR-3 of SEQ ID NO: 12; a VL CDR-1 of SEQ ID NO: 22; a VL CDR-2 of SEQ ID NO: 23, and a VL CDR-3 of SEQ ID NO: 24.

In some embodiments, the immunocapture reagent is an antibody or antigen-binding fragment which comprises a VH of SEQ ID NO: 25 and a VL of SEQ ID NO: 29. In some embodiments, the immunocapture reagent is an antibody or antigen-binding fragment which comprises a VH of SEQ ID NO: 26 and a VL of SEQ ID NO: 30. In some embodiments, the immunocapture reagent is an antibody or antigen-binding fragment which comprises a VH of SEQ ID NO: 27 and VL of SEQ ID NO: 31. In some embodiments, the immunocapture reagent is an antibody or antigen-binding fragment which comprises a VH of SEQ ID NO: 28 and a VL of SEQ ID NO: 32. In some embodiments, the immunocapture reagent is an antibody or antigen-binding fragment which comprises a VH having at least 90% identity to the sequence of SEQ ID NO: 25 and a VL having at least 90% identity to the sequence of SEQ ID NO: 29. In some embodiments, the immunocapture reagent is an antibody or antigen-binding fragment which comprises a variable heavy chain (VH) having at least 95% identity to the sequence of SEQ ID NO: 25 and a variable light chain (VL) having at least 95% identity to the sequence of SEQ ID NO: 29. In some embodiments, the immunocapture reagent is an antibody or antigen-binding fragment which comprises a variable heavy chain (VH) having at least 90% identity to the sequence of SEQ ID NO: 26 and a variable light chain (VL) having at least 90% identity to the sequence of SEQ ID NO: 30. In some embodiments, the immunocapture reagent is an antibody or antigen-binding fragment which comprises a variable heavy chain (VH) having at least 95% identity to the sequence of SEQ ID NO: 26 and a variable light chain (VL) having at least 95% identity to the sequence of SEQ ID NO: 30. In some embodiments, the immunocapture reagent is an antibody or antigen-binding fragment which comprises antibody or antigen-binding fragment which comprises a variable heavy chain (VH) having at least 90% identity to the sequence of SEQ ID NO: 27 and a variable light chain (VL) having at least 90% identity to the sequence of SEQ ID NO: 31. In some embodiments, the immunocapture reagent is an antibody or antigen-binding fragment which comprises a variable heavy chain (VH) having at least 95% identity to the sequence of SEQ ID NO: 27 and a variable light chain (VL) having at least 95% identity to the sequence of SEQ ID NO: 31. In some embodiments, the immunocapture reagent is an antibody or antigen-binding fragment which comprises a variable heavy chain (VH) having at least 90% identity to the sequence of SEQ ID NO: 28 and a variable light chain (VL) having at least 90% identity to the sequence of SEQ ID NO: 32. In some embodiments, the immunocapture reagent is an antibody or antigen-binding fragment which comprises a variable heavy chain (VH) having at least 95% identity to the sequence of SEQ ID NO: 28 and a variable light chain (VL) having at least 95% identity to the sequence of SEQ ID NO: 32.

In one embodiment, the immunocapture reagent is an antibody or antigen-binding fragment which comprises a heavy chain having the sequence of SEQ ID NO: 33 and a light chain having the sequence of SEQ ID NO: 37. In another embodiment, the immunocapture reagent is an antibody or antigen-binding fragment which comprises a heavy chain having the sequence of SEQ ID NO: 34 and a light chain having the sequence of SEQ ID NO: 38. In another embodiment, the immunocapture reagent is an antibody or antigen-binding fragment which comprises a heavy chain having the sequence of SEQ ID NO: 35 and a light chain having the sequence of SEQ ID NO: 39. In another embodiment, the immunocapture reagent is an antibody or antigen-binding fragment which comprises a heavy chain of SEQ ID NO: 36 and a light chain of SEQ ID NO: 40.

In some embodiments, the immunocapture reagent is an antibody or antigen-binding fragment which binds to human FOLR1 with a Kd of about 1.0 nM to about 10 nM. In other embodiments, the immunocapture reagent is an antibody or antigen-binding fragment which binds to human FOLR1 with a Kd of about 0.5 nM to about 5 nM.

In some embodiments, the immunocapture reagent is biotinylated. In some embodiments, the immunocapture reagent is bound to the solid support through a biotin-streptavidin interaction. In some embodiments, the solid support is a mass spectrometric immunoassay (MSIA) microcolum. In other embodiments, the immunocapture reagent comprises magnetic beads.

To perform the initial immunocapture step, a sample containing FOLR1 is incubated with the immunocapture reagent bound to a solid support. A wash step may be performed after the immunocapture step to further purify the captured FOLR1. In some embodiments, one or more wash steps are performed following incubation of the sample with the immunocapture reagent and prior to elution of the captured FOLR1. In some embodiments, two or more wash steps are performed prior to elution of the captured FOLR1. In some embodiments, at least two, at least three, at least four, at least five, at least six, at least seven, at least eight, at least nine, or at least ten wash steps are performed on the captured FOLR1. In some embodiments, the wash step comprises contacting the captured FOLR1 with washing buffers. In some embodiments, the washing buffers are commercially available washing buffers. In some embodiments, the wash step comprises contacting the captured FOLR1 with a salt solution and a detergent. In some embodiments, the salt solution is 400 mM NaCl and the detergent is 0.1% Tween 20.

Following immunocapture, the FOLR1 is released from the solid support by performing an elution step. In some embodiments, the elution step is performed by contacting the captured FOLR1 with an acidic solution. In some embodiments, the acidic solution is a commercially available solution. In some embodiments, the eluate is brought to neutral pH by addition of a neutralization buffer. In some embodiments, the neutralization buffer is 500 mM Ammonium Bicarbonate at pH 8. In some embodiments, the neutralization buffer is a commercially available buffer.

b. Liquid Chromatography-Mass Spectrometry

Following immunocapture and elution, the resulting FOLR1 protein is digested into peptides and analyzed by liquid chromatography-mass spectrometry (LC/MS). In some embodiments, the FOLR1-containing solution is alkylated and reduced prior to analysis by LC/MS. In some embodiments, the FOLR1 is alkylated with methanol. In some embodiments, the FOLR1 is reduced by contacting the FOLR1 with a solution containing tris(2-carboxyethyl)phosphine (TCEP). In some embodiments, a solution containing 100 mM TCEP is used to reduce the FOLR1. In some embodiments, a cysteine blocking reagent is added to the FOLR1-containing solution after alkylation and reduction. In some embodiments, the cysteine blocking reagent is iodoacetamide (IAM). In some embodiments, a solution containing 100 mM IAM is added to the FOLR1-containing solution.

After alkylation and reduction, the FOLR1 is digested into peptides. In some embodiments, the FOLR1 is digested with trypsin. In some embodiments, the FOLR1 is digested with Lys-C. In some embodiments, the FOLR1 is digested with a mixture of trypsin and Lys-C. In some embodiments, the FOLR1 is digested by contacting the FOLR1 with a 50 mM ammonium bicarbonate solution containing 30 ng/μL trypsin/Lys-C.

Digestion of the FOLR1 with trypsin/Lys-C produces peptides that are useful in conducting quantitative analysis of the sample by LC/MS. In some embodiments, isolated signature peptides of the invention are provided in the digestion product of FOLR1. Signature peptides produced by digestion of FOLR1 are provided in Table 7 below.

TABLE 7
FOLR1 signature peptide amino acid sequences

| | |
|---|---|
| sFRα Signature | TELLNVCMNAK |
| Peptide Sequence 1 | (SEQ ID NO: 42) |
| sFRα Signature | IAWAR |
| Peptide Sequence 2 | (SEQ ID NO: 43) |
| sFRα Signature | VLNVPLCK |
| Peptide Sequence 3 | (SEQ ID NO: 44) |
| sFRα Signature | CIQMWFDPAQGNPNEEVAR |
| Peptide Sequence 4 | (SEQ ID NO: 45) |

After digestion of FOLR1 into peptides, the peptide-containing solution is prepared for LC/MS analysis. In some embodiments, a surfactant is added to the peptide-containing solution prior to LC/MS analysis. In some embodiments, the surfactant is a commercial reagent formulated for mass spectrometry analysis. In some embodiments, the reaction with surfactant is quenched by adding 10% formic acid.

Following digestion and preparation of the samples for LC/MS analysis, the samples are injected into an LC/MS instrument and analyzed. In some embodiments, at least two signature peptides of FOLR1 are selected and monitored at the LC/MS analysis step. In some embodiments, at least three signature peptides of FOLR1 are selected and monitored at the LC/MS analysis step. In some embodiments, at least four signature peptides of FOLR1 are selected and monitored at the LC/MS analysis step. In some embodiments, the at least four signature peptides selected and monitored at the LC/MS step comprise: a peptide having the amino acid sequence of SEQ ID NO: 42; a peptide having the amino acid sequence of SEQ ID NO: 43; a peptide having the amino acid sequence of SEQ ID NO: 44; and a peptide having the amino acid sequence of SEQ ID NO: 45. In some embodiments, quantitative measurements of the FOLR1 levels are provided by LC/MS analysis. In further embodiments, the level of FOLR1 in the sample is quantitated by comparing the level of FOLR1 in the sample to a reference level of FOLR1. Methods of analysis by LC/MS analysis are well known in the art and are described in, e.g., Yang et al. Scientific Reports. 2015 Nov. 17; 5:16733; doi10.1038/srep16733.

In some embodiments, the detection of FOLR1 in the sample is not inhibited by the presence of IMGN853 in the sample. In some embodiments, the detection of FOLR1 in the sample is not inhibited by the presence of huMov19 in the sample. In some embodiments, the detection of FOLR1 in the sample is not inhibited by the presence of an antibody or antigen-binding fragment present in the sample, wherein said antibody or antigen-binding fragment comprises: a variable light chain (VL) complementarity determining region (CDR)-1 of SEQ ID NO: 59; a VL CDR-2 of SEQ ID NO: 60; a VL CDR-3 of SEQ ID NO: 61; a variable heavy chain (VH) CDR-1 of SEQ ID NO: 62; a VH CDR-2 of SEQ ID NO: 64; and a VH CDR-3 of SEQ ID NO: 65. In some embodiments, the detection of FOLR1 in the sample is not inhibited by the presence of an antibody or antigen-binding fragment present in the sample, wherein said antibody or antigen-binding fragment comprises a VH having the sequence of SEQ ID NO: 56 and a VL having the sequence of SEQ ID NO: 57 or SEQ ID NO: 58. In some embodiments, the detection of FOLR1 is not inhibited by the presence of an antibody or antigen-binding fragment present in the sample, wherein said antibody or antigen-binding fragment comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774. In some embodiments, the detection of FOLR1 in the sample is not inhibited by the presence of folic acid in the sample.

In some embodiments, at least 0.5 ng/mL FORL1 can be detected in a sample by performing the methods of the invention. In some embodiments, at least 0.3 ng/mL FOLR1 can be detected in a sample by performing the methods of the invention. In some embodiments, at least 0.25 ng/mL FOLR1 can be detected in a sample by performing the methods of the invention. In some embodiments, at least 0.2 ng/mL FOLR1 can be detected in a sample by performing the methods of the invention. In some embodiments, at least 0.15 ng/mL FOLR1 can be detected in a sample by performing the methods of the invention. In some embodiments, a signal-to-noise ratio of at least 5 is observed by performing the methods of the invention. In some embodiments, a signal-to-noise ratio of at least 6 is observed by performing the methods of the invention. In some embodiments, a signal-to-noise ratio of at least 7 is observed by performing the methods of the invention. In some embodiments, a signal-to-noise ratio of at least 8 is observed by performing the methods of the invention. In some embodiments, a signal-to-noise ratio of at least 9 is observed by performing the methods of the invention. In some embodiments, a signal-to-noise ratio of at least 10 is observed by performing the methods of the invention.

### IV. Kits

As a matter of convenience, the assay method of this invention can be provided in the form of a kit. Such a kit is a packaged combination including the basic elements of: (a) a first reagent, which can be an immunocapture reagent, which binds to FOLR1; and (b) a digestion reagent, which can digest captured FOLR1 into peptides. The kit may further comprise at least one peptide derived from FOLR1. These basic elements are defined hereinabove and in the Examples below.

In some embodiments, the immunocapture reagent is an antibody or antigen-binding fragment which binds to FOLR1. In a further embodiment, the immunocapture reagent is an antibody or antigen-binding fragment which comprises a variable heavy chain (VH) complementarity determining region (CDR)-1 of SEQ ID NO: 1; a VH CDR-2 of SEQ ID NO: 2; a VH CDR-3 of SEQ ID NO: 3; a variable light chain (VL) complementarity determining region (CDR)-1 of SEQ ID NO: 13; a VL CDR-2 of SEQ ID NO: 14, and a VL CDR-3 of SEQ ID NO: 15. In other embodiments, the immunocapture reagent is an antibody or antigen-binding fragment which comprises a VH CDR-1 of SEQ ID NO: 4; a VH CDR-2 of SEQ ID NO: 5; a VH CDR-3 of SEQ ID NO: 6; a VL CDR-1 of SEQ ID NO: 16; a VL CDR-2 of SEQ ID NO: 17, and a VL CDR-3 of SEQ ID NO: 18. In other embodiments, the immunocapture reagent is an antibody or antigen-binding fragment which comprises a VH CDR-1 of SEQ ID NO: 7; a VH CDR-2 of SEQ ID NO: 8; a VH CDR-3 of SEQ ID NO: 9; a VL CDR-1 of SEQ ID NO: 19; a VL CDR-2 of SEQ ID NO: 20, and a VL CDR-3 of SEQ ID NO: 21. In other embodiments, the immunocapture reagent is an antibody or antigen-binding fragment which comprises a VH CDR-1 of SEQ ID NO: 10; a VH CDR-2 of SEQ ID NO: 11; a VH CDR-3 of SEQ ID NO: 12; a VL CDR-1 of SEQ ID NO: 22; a VL CDR-2 of SEQ ID NO: 23, and a VL CDR-3 of SEQ ID NO: 24.

In some embodiments, the immunocapture reagent is an antibody or antigen-binding fragment which comprises a VH of SEQ ID NO: 25 and a VL of SEQ ID NO: 29. In other embodiments, the immunocapture reagent is an antibody or antigen-binding fragment which comprises a VH of SEQ ID NO: 26 and a VL of SEQ ID NO: 30. In some embodiments, the immunocapture reagent is an antibody or antigen-binding fragment which comprises a VH of SEQ ID NO: 27 and VL of SEQ ID NO: 31. In some embodiments, the immunocapture reagent is an antibody or antigen-binding fragment which comprises a VH of SEQ ID NO: 28 and a VL of SEQ ID NO: 32. In some embodiments, the immunocapture reagent comprises an antibody comprising a VH with at least 90% identity to any one of SEQ ID NOs: 25-28. In some embodiments, the immunocapture reagent comprises an antibody comprising a VH with at least 95% identity to any one of SEQ ID NOs: 25-28. In some embodiments, the immunocapture reagent comprises an antibody comprising a VL with at least 90% identity to any one of SEQ ID NOs: 29-32. In some embodiments, the immunocapture reagent comprises an antibody comprising a VL with at least 95% identity to any one of SEQ ID NOs: 29-32.

In some embodiments, the immunocapture reagent is an antibody or antigen-binding fragment which comprises a heavy chain having the sequence of SEQ ID NO: 33 and a light chain having the sequence of SEQ ID NO: 37. In other embodiments, the immunocapture reagent is an antibody or antigen-binding fragment which comprises a heavy chain having the sequence of SEQ ID NO: 34 and a light chain having the sequence of SEQ ID NO: 38. In other embodiments, the immunocapture reagent is an antibody or antigen-binding fragment which comprises a heavy chain having the sequence of SEQ ID NO: 35 and a light chain having the sequence of SEQ ID NO: 39. In other embodiments, the immunocapture reagent is an antibody or antigen-binding fragment which comprises a heavy chain of SEQ ID NO: 36 and a light chain of SEQ ID NO: 40.

In some embodiments, the immunocapture reagent is muFR1-9. In other embodiments, the immunocapture reagent is muFR1-13. In some embodiments, the immunocapture reagent is an antibody or antigen-binding fragment which binds to human FOLR1 with a Kd of about 1.0 nM to about 10 nM. In other embodiments, the immunocapture reagent is an antibody or antigen-binding fragment which binds to human FOLR1 with a Kd of about 0.5 nM to about 5 nM. In some embodiments, the immunocapture reagent is biotinylated.

In some embodiments, the kit further comprises a solid support for the capture reagents, which can be provided as a separate element or upon which the capture reagents are already immobilized. Hence, the capture antibodies in the kit can be immobilized on a solid support, or they can be immobilized on such support that is included with the kit or provided separately from the kit. In some embodiments, the solid support comprises a mass spectrometric immunoassay (MSIA) microcolum. In other embodiments, the solid support comprises magnetic beads. In some embodiments, the solid support is coated with streptavidin. In some embodiments, the immunocapture reagent is attached to the solid support through a biotin-streptavidin interaction.

In some embodiments, the kit further contains at least one FOLR1 peptide, which can be used as a standard in liquid chromatography-mass spectrometry analyses. In some embodiments, the kit contains a peptide comprising the sequence of SEQ ID NO: 42. In some embodiments, the kit contains a peptide comprising the sequence of SEQ ID NO: 43. In some embodiments, the kit contains a peptide comprising the sequence of SEQ ID NO: 44. In some embodiments, the kit contains a peptide comprising the sequence of SEQ ID NO: 45. In one embodiment, the kit comprises four signature peptides which can be used as a standard in liquid chromatography-mass spectrometry analyses. In one embodiment, the four signature peptides consist of 1) a peptide comprising the sequence of SEQ ID NO: 42; 2) a peptide comprising the sequence of SEQ ID NO: 43; 3) a peptide comprising the sequence of SEQ ID NO: 44; and 4) a peptide comprising the sequence of SEQ ID NO: 45.

The kit also typically contains instructions for carrying out the assay, and/or FOLR1 protein or FOLR1 peptides to serve as a standard, as well as other additives such as stabilizers, washing and incubation buffers, and the like. The kit can also include instructions for quantitative measurement of FOLR1 levels in a sample.

The components of the kit will be provided in predetermined ratios, with the relative amounts of the various reagents suitably varied to provide for concentrations in solution of the reagents that substantially maximize the sensitivity of the assay. Particularly, the reagents can be provided as dry powders, usually lyophilized, including excipients, which on dissolution will provide for a reagent solution having the appropriate concentration for combining with the sample to be tested.

Embodiments of the present disclosure can be further defined by reference to the following non-limiting examples, which describe in detail the methods of the present disclosure. It will be apparent to those skilled in the art that many modifications, both to materials and methods, can be practiced without departing from the scope of the present disclosure.

### EXAMPLES

It is understood that the examples and embodiments described herein are for illustrative purposes only and that various modifications or changes in light thereof will be suggested to persons skilled in the art and are to be included within the spirit and purview of this application.

### Example 1

### sFRα Immunocapture-LC/MS Assay

An ELISA-based assay was developed and described in WO 2014/036495. This assay was performed using ELISA plates coated with FOLR1-Fc (fusion peptide of huFOLR1 and murine IgG2A hinge, CH2, CH3), muFR1-9 as the capture antibody, and biotinylated muFR1-13. The assay was optimized by a systematic approach with the criteria being reproducible signals with a high signal to noise ratio, minimal matrix effects in human plasma samples, high repeatability and precision, and lowest limit of detection. The optimal ELISA conditions chosen resulted in detection of sFRα as low as 25 ng/mL with neglibible/low noise. Despite the optimized conditions, this ELISA assay did not provide enough sensitivity to distinguish between sFRα levels in normal subjects versus the range of distribution of sFRα levels in ovarian cancer patients to allow correlative analysis of sFRα levels and the disease state.

A more recent ELISA-based assay using two different human FRa-specific mouse monoclonal antibodies reported in Kurosaki et al., Int. J. Cancer. 2016 Apr. 15; 138(8):1994-2002 was developed to investigate the levels of sFRα in serum of patients clinically suspected of having malignant ovarian tumors and to evaluate sFRα as a diagnostic marker for ovarian cancer. This ELISA assay had a dynamic range of 250-8000 pg/mL and a signal-to-noise ratio of 3.4 for the lowest calibrator (250 pg/mL). While this ELISA assay provided a more sensitive dynamic range of sFRα detection, ELISA readouts are typically measured spectrophotometrically at 450 nm and corrected for absorbance at 620 nm and therefore are not as selective and likely to produce more false positive result.

The assays of the present invention were developed which fulfilled the criteria of both sensitivity and selectivity. The accurate measurement of signature peptides by the LC/MS step provides a second level of selectivity not provided by typical ELISA methods. Further, the sensitivity and selectivity of detecting sFRα afforded by the immunocapture-LC/MS assay also makes this assay useful in exploring sFRα as a predictive and/or prognostic biomarker for ovarian cancer patients.

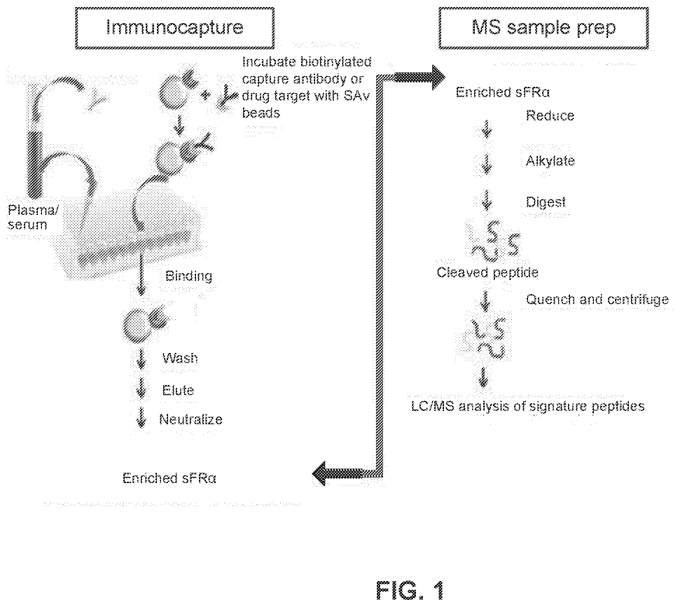

The assay as shown in FIG. 1 includes the steps of incubating biotinylated capture antibody (e.g., an antibody of the invention, such as, the muFR1-9 or muFR1-13 antibody) solution with streptavidin-coated immunocapture media (MSIA microcolumns or magnetic beads), incubating samples with immunocapture media, washing the immunocapture media with washing buffers, salt solution, and detergent in multiple washes, discarding the supernatant, releasing of sFRα from immunocapture media with an acidic solution, and neutralizing the sFRα solution followed by reduction and alkylation. The samples are then digested with Trypsin/Lys-C and injected to LC/MS for analysis. Four different signature peptides of sFRα were selected and monitored at the LC/MS analysis step. Specifically, the peptide of SEQ ID NO: 42 was used for reporting the protein concentration, and peptides of SEQ ID NOs: 43-45 were used for confirmation.

Optimization steps to improve assay performance during sample preparation include modifying conditions to improve immunocapture efficiency, washing procedures to remove potential interference from plasma matrix, and modifying conditions to improve enzymatic digestion efficiency. Optimization steps during LC/MS include identifying a signature peptide sequence that provides good chromatographic separation and mass spectrometric response. Automation options for immunocapture were evaluated with MSIA microcolumn technology and magnetic bead processing. Both options were compatible with use of 96-well plates to significantly improve sample throughput and similar sensitivity was observed for both automation platforms. Evaluation was also performed on the assay using either antigen-depleted plasma or surrogate matrix.

The working assay parameters are as follows: sample processing including immunocapture and enzymatic digestion of 8.5 hours, LC/MS analysis of 10 minutes per sample, calibration standards prepared in 5% BSA, PBS (surrogate matrix, 0.65 ng/mL to 40.63 ng/mL), sample volume at 0.3 mL, S/N>10 for four signature peptides at LLOQ level, STD acceptance criteria of ±20% of nominal value except for LLOQ (±25%), QC acceptance criteria of ±20% of nominal value.

### Example 2

### Data Analysis of Human Plasma Samples Using the sFRα Immunocapture-LC/MS Assay

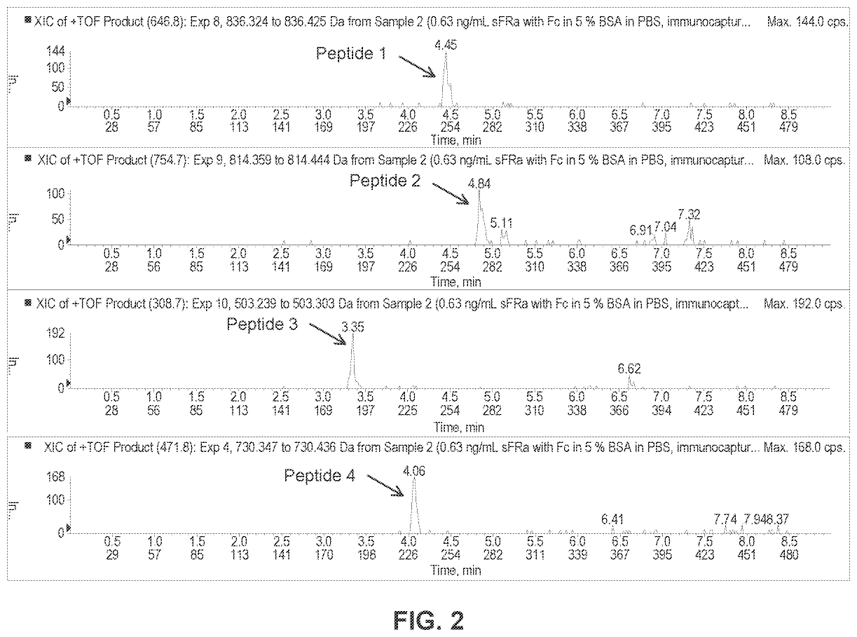

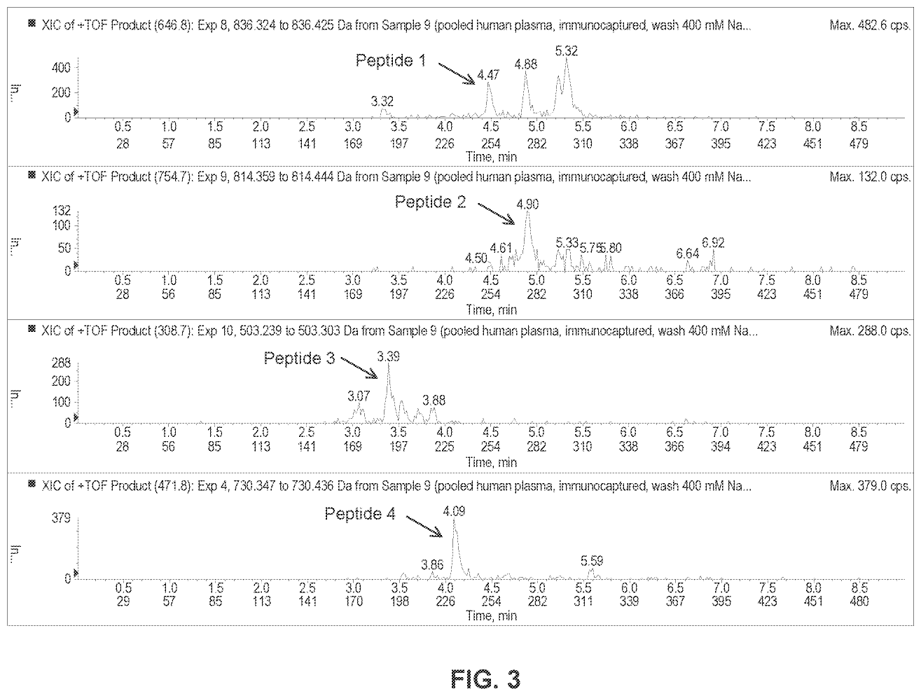

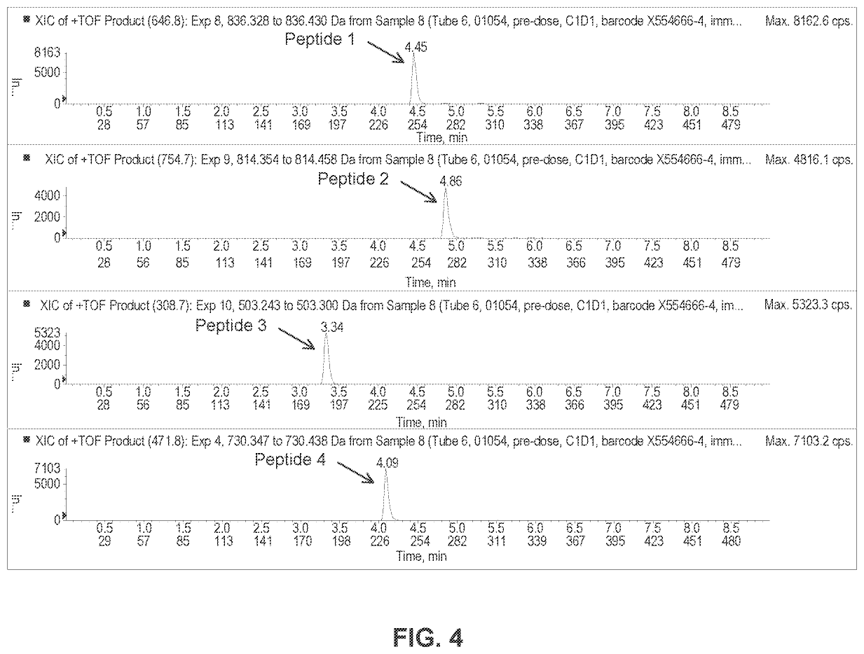

Experiments were designed using the above parameters to measure the levels of sFRα in a sample containing low level of sFRα in surrogate matrix at 0.3 ng/mL. As shown in FIG. 2, the results of these experiments demonstrate that very low levels of sFRα can be measured by the immunocapture-LC/MS assay. Experiments were also designed to measure the levels of sFRα in a normal human plasma sample. As shown in FIG. 3, endogenous levels of sFRα, found around 0.5 ng/mL, can be measured by Immunocapture-LC/MS Assay. FIG. 4 shows an extracted ion chromatogram from a patient pre-dose sample with elevated sFRα. The results of this experiment demonstrates that patient plasma samples with elevated levels of sFRα can be clearly distinguished from that of endogenous levels of normal human plasma sample.

Experiments were designed to test the assay tolerability in the presence of a FRa-targeting immunoconjugate, IMGN853, in plasma samples. sFRα QC samples were prepared in plasma, at low, mid, and high levels, spiked with IMGN853 at projected Cmax concentration, and compared to sFRα QC samples prepared in plasma without IMGN853. No interference was observed in either sets of QC samples, and both sets of samples have similar responses.

The results of these experiments show that the immunocapture-LC/MS assay of the invention has increased sensitivity (0.3 ng/mL, signal-to-noise ratio of at least 10, i.e., 2-fold more sensitive than the ELISA assays in the art) and enhanced selectivity to measure sFRα in human plasma samples in the presence and absence of a FRa-targeting immunoconjugate.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

What is claimed is:

1. A method of detecting human folate receptor 1 (FOLR1) in a sample comprising:
(a) capturing said folate receptor 1 (FOLR1) with an immunocapture reagent bound to a solid support;
(b) eluting FOLR1 from the solid support;
(c) digesting the eluted FOLR1; and
(d) performing liquid chromatography-mass spectrometry (LC/MS) analysis on the digested FOLR1, wherein said FOLR1 is detected by monitoring the chromatographic separation and mass spectrometric response of at least one signature FOLR1 peptide.

2. The method of claim 1, wherein the level of FOLR1 in the sample is quantitated by said LC/MS analysis.

3. The method of claim 2, wherein the level of FOLR1 in the sample is quantitated by comparing the level of FOLR1 in the sample to a reference level of FOLR1.

4. The method of any one of claims 1-3, wherein the immunocapture reagent comprises an antibody or antigen-binding fragment which binds to FOLR1.

5. The method of claim 4, wherein binding of the antibody or antigen-binding fragment to FOLR1 is not competitively inhibited by binding of IMGN853 to FOLR1.

6. The method of claim 4, wherein binding of the antibody or antigen-binding fragment to FOLR1 is not competitively inhibited by binding of huMov19 to FOLR1.

7. The method of claim 4, wherein binding of the antibody or antigen-binding fragment to FOLR1 is not competitively inhibited by binding of a second antibody or antigen-binding fragment to FOLR1, wherein the second antibody or antigen-binding fragment comprises: a variable light chain (VL) complementarity determining region (CDR)-1 of SEQ ID NO: 59; a VL CDR-2 of SEQ ID NO: 60; a VL CDR-3 of SEQ ID NO: 61; a variable heavy chain (VH) CDR-1 of SEQ ID NO: 62; a VH CDR-2 of SEQ ID NO: 64; and a VH CDR-3 of SEQ ID NO: 65.

8. The method of claim 4, wherein binding of the antibody or antigen-binding fragment to FOLR1 is not competitively inhibited by binding of a second antibody or antigen-binding fragment to FOLR1, wherein the second antibody or antigen-binding fragment comprises a variable heavy chain (VH) having the sequence of SEQ ID NO: 56 and a variable light chain (VL) having the sequence of SEQ ID NO: 57 or SEQ ID NO: 58.

9. The method of claim 4, wherein binding of the antibody or antigen-binding fragment to FOLR1 is not competitively inhibited by binding of a second antibody or antigen-binding fragment to FOLR1, wherein the second antibody or antigen-binding fragment comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774.

10. The method of claim 4, wherein binding of the antibody or antigen-binding fragment to FOLR1 is not inhibited by binding of folic acid to FOLR1.

11. The method of claim 4, wherein the antibody is muFR1-9.

12. The method claim 4, wherein the antibody is muFR1-13.

13. The method of claim 4, wherein the antibody or antigen-binding fragment comprises a variable heavy chain (VH) complementarity determining region (CDR)-1 of SEQ ID NO: 1; a VH CDR-2 of SEQ ID NO: 2; a VH CDR-3 of SEQ ID NO: 3; a variable light chain (VL) complementarity determining region (CDR)-1 of SEQ ID NO: 13; a VL CDR-2 of SEQ ID NO: 14, and a VL CDR-3 of SEQ ID NO: 15.

14. The method of claim 4, wherein the antibody or antigen-binding fragment comprises a variable heavy chain (VH) complementarity determining region (CDR)-1 of SEQ ID NO: 4; a VH CDR-2 of SEQ ID NO: 5; a VH CDR-3 of SEQ ID NO: 6; a variable light chain (VL) complementarity determining region (CDR)-1 of SEQ ID NO: 16; a VL CDR-2 of SEQ ID NO: 17, and a VL CDR-3 of SEQ ID NO: 18.

15. The method of claim 4, wherein the antibody or antigen-binding fragment comprises a variable heavy chain (VH) having at least 90% identity to the sequence of SEQ ID NO: 25 and a variable light chain (VL) having at least 90% identity to the sequence of SEQ ID NO: 29.

16. The method of claim 4, wherein the antibody or antigen-binding fragment comprises a variable heavy chain (VH) having at least 95% identity to the sequence of SEQ ID NO: 25 and a variable light chain (VL) having at least 95% identity to the sequence of SEQ ID NO: 29.

17. The method of claim 4, wherein the antibody or antigen-binding fragment comprises a variable heavy chain (VH) having at least 90% identity to the sequence of SEQ ID NO: 26 and a variable light chain (VL) having at least 90% identity to the sequence of SEQ ID NO: 30.

18. The method of claim 4, wherein the antibody or antigen-binding fragment comprises a variable heavy chain (VH) having at least 95% identity to the sequence of SEQ ID NO: 26 and a variable light chain (VL) having at least 95% identity to the sequence of SEQ ID NO: 30.

19. The method of claim 4, wherein the antibody or antigen-binding fragment comprises a variable heavy chain (VH) having the sequence of SEQ ID NO: 25 and a variable light chain (VL) having the sequence of SEQ ID NO: 29.

20. The method of claim 4, wherein the antibody or antigen-binding fragment comprises a variable heavy chain (VH) having the sequence of SEQ ID NO: 26 and a variable light chain (VL) having the sequence of SEQ ID NO: 30.

21. The method of claim 4, wherein the antibody or antigen-binding fragment comprises a heavy chain having the sequence of SEQ ID NO: 33 and a light chain having the sequence of SEQ ID NO: 37.

22. The method of claim 4, wherein the antibody or antigen-binding fragment comprises a heavy chain having the sequence of SEQ ID NO: 34 and a light chain having the sequence of SEQ ID NO: 38.

23. The method of claim 4, wherein the antibody or antigen-binding fragment binds to human FOLR1 with a Kd of about 1.0 nM to about 10 nM.

24. The method of claim 4, wherein the antibody or antigen-binding fragment binds to human FOLR1 with a Kd of about 0.5 nM to about 5 nM.

25. The method of any one of claims 1-24, wherein the solid support comprises a mass spectrometric immunoassay (MSIA) microcolumn.

26. The method of any one of claims 1-24, wherein the solid support comprises magnetic beads.

27. The method of any one of claims 1-26, wherein at least one wash step is performed prior to eluting FOLR1 from the solid support.

28. The method of claim 27, wherein two or more wash steps are performed prior to eluting FOLR1 from the solid support.

29. The method of claim 27 or 28, where the wash step comprises contacting the FOLR1 bound to the solid support with washing buffers, a salt solution, and a detergent.

30. The method of any one of claims 1-29, wherein FOLR1 is eluted from the solid support with an acidic solution.

31. The method of claim 30, wherein the FOLR1 is reduced and alkylated prior to digesting the FOLR1.

32. The method of any one of claims 1-31, wherein FOLR1 is digested with Trypsin/Lys-C.

33. The method of any one of claims 1-32, wherein digesting the FOLR1 produces a peptide comprising the sequence of SEQ ID NO: 42.

34. The method of any one of claims 1-33, wherein digesting the FOLR1 produces a peptide comprising the sequence of SEQ ID NO: 43.

35. The method of any one of claims 1-34, wherein digesting the FOLR1 produces a peptide comprising the sequence of SEQ ID NO: 44.

36. The method of any one of claims 1-35, wherein digesting the FOLR1 produces a peptide comprising the sequence of SEQ ID NO: 45.

37. The method of any one of claims 1-36, wherein at least two signature peptides of FOLR1 are selected and monitored at the LC/MS analysis step.

38. The method of any one of claims 1-37, wherein at least three signature peptides of FOLR1 are selected and monitored at the LC/MS analysis step.

39. The method of any one of claims 1-38, wherein at least four signature peptides of FOLR1 are selected and monitored at the LC/MS analysis step.

40. The method of claim 39, wherein the signature peptides comprise:
(a) a peptide comprising the sequence of SEQ ID NO: 42;
(b) a peptide comprising the sequence of SEQ ID NO: 43;
(c) a peptide comprising the sequence of SEQ ID NO: 44; and
(d) a peptide comprising the sequence of SEQ ID NO: 45.

41. The method of any one of claims 1-40, wherein said sample comprises a bodily fluid.

42. The method of claim 41, wherein said bodily fluid is plasma.

43. The method of claim 41, wherein said bodily fluid is serum.

44. The method of claim 41, wherein said bodily fluid is ascites fluid.

45. The method of any one of claims 1-40, wherein the sample comprises a peripheral blood sample.

46. The method of any one of claims 1-45, wherein the sample is obtained from a patient having cancer.

47. The method of claim 46, wherein said cancer is selected from the group consisting of: ovarian, brain, breast, uterine, endometrial, pancreatic, renal, lung cancer, and cancer of the peritoneum.

48. The method of claim 47, wherein the cancer is ovarian cancer.

49. The method of any one of claims 1-48, wherein detecting FOLR1 is not inhibited by IMGN853 present in the sample.

50. The method of any one of claims 1-48, wherein detecting FOLR1 is not inhibited by huMov19 present in the sample.

51. The method of any one of claims 1-48, wherein detecting FOLR1 is not inhibited by an antibody or antigen-binding fragment present in the sample, wherein said antibody or antigen-binding fragment comprises: a variable light chain (VL) complementarity determining region (CDR)-1 of SEQ ID NO: 59; a VL CDR-2 of SEQ ID NO: 60; a VL CDR-3 of SEQ ID NO: 61; a variable heavy chain (VH) CDR-1 of SEQ ID NO: 62; a VH CDR-2 of SEQ ID NO: 64; and a VH CDR-3 of SEQ ID NO: 65.

52. The method of any one of claims 1-48, wherein detecting FOLR1 is not inhibited by an antibody or antigen-binding fragment present in the sample, wherein said antibody or antigen-binding fragment comprises a variable heavy chain (VH) having the sequence of SEQ ID NO: 56 and a variable light chain (VL) having the sequence of SEQ ID NO: 57 or SEQ ID NO: 58.

53. The method of any one of claims 1-48, wherein detecting FOLR1 is not inhibited by an antibody or antigen-binding fragment present in the sample, wherein said antibody or antigen-binding fragment comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774.

54. The method of any one of claims 1-53, wherein detecting FOLR1 is not inhibited by folic acid present in the sample.

55. The method of any one of claims 1-54, which can detect at least 0.5 ng/mL FOLR1 in a sample.

56. The method of any one of claims 1-54, which can detect at least 0.3 ng/mL FOLR1 in a sample.

57. The method of any one of claims 1-54, which can detect at least 0.25 ng/mL FOLR1 in a sample.

58. The method of any one of claims 1-57, wherein the signal-to-noise ratio is at least 5.

59. The method of any one of claims 1-57, wherein the signal-to-noise ratio is at least 10.

60. The method of any one of claims 1-59, wherein the FOLR1 is shed FOLR1.

61. A peptide consisting of the sequence of SEQ ID NO: 42.

62. A peptide consisting of the sequence of SEQ ID NO: 43.

63. A peptide consisting of the sequence of SEQ ID NO: 44.

64. A peptide consisting of the sequence of SEQ ID NO: 45.

65. A kit comprising: an immunocapture reagent which binds to FOLR1, a digestion reagent, and at least one peptide selected from the group consisting of:
(a) a peptide comprising the sequence of SEQ ID NO: 42;
(b) a peptide comprising the sequence of SEQ ID NO: 43;
(c) a peptide comprising the sequence of SEQ ID NO: 44; and
(d) a peptide comprising the sequence of SEQ ID NO: 45.

66. The kit of claim 65, wherein the immunocapture reagent comprises an antibody or antigen-binding fragment which binds to FOLR1.

67. The kit of claim 66, wherein the antibody is muFR1-9.

68. The kit of claim 66, wherein the antibody is muFR1-13.

69. The kit of claim 66, wherein the antibody or antigen-binding fragment comprises a variable heavy chain (VH) complementarity determining region (CDR)-1 of SEQ ID NO: 1; a VH CDR-2 of SEQ ID NO: 2; a VH CDR-3 of SEQ ID NO: 3; a variable light chain (VL) complementarity determining region (CDR)-1 of SEQ ID NO: 13; a VL CDR-2 of SEQ ID NO: 14, and a VL CDR-3 of SEQ ID NO: 15.

70. The kit of claim 66, wherein the antibody or antigen-binding fragment comprises the variable heavy chain (VH) complementarity determining region (CDR)-1 of SEQ ID NO: 4; a VH CDR-2 of SEQ ID NO: 5; a VH CDR-3 of SEQ ID NO: 6; a variable light chain (VL) complementarity determining region (CDR)-1 of SEQ ID NO: 16; a VL CDR-2 of SEQ ID NO: 17, and a VL CDR-3 of SEQ ID NO: 18.

71. The kit of claim 66, wherein the antibody or antigen-binding fragment comprises a variable heavy chain (VH) having at least 90% identity to the sequence of SEQ ID NO: 25 and a variable light chain (VL) having at least 90% identity to the sequence of SEQ ID NO: 29.

72. The kit of claim 66, wherein the antibody or antigen-binding fragment comprises a variable heavy chain (VH) having at least 95% identity to the sequence of SEQ ID NO: 25 and a variable light chain (VL) having at least 95% identity to the sequence of SEQ ID NO: 29.

73. The kit of claim 66, wherein the antibody or antigen-binding fragment comprises a variable heavy chain (VH) having at least 90% identity to the sequence of SEQ ID NO: 26 and a variable light chain (VL) having at least 90% identity to the sequence of SEQ ID NO: 30.

74. The kit of claim 66, wherein the antibody or antigen-binding fragment comprises a variable heavy chain (VH) having at least 95% identity to the sequence of SEQ ID NO: 26 and a variable light chain (VL) having at least 95% identity to the sequence of SEQ ID NO: 30.

75. The kit of claim 66, wherein the antibody or antigen-binding fragment comprises a variable heavy chain (VH) having the sequence of SEQ ID NO: 25 and a variable light chain (VL) having the sequence of SEQ ID NO: 29.

76. The kit of claim 66, wherein the antibody or antigen-binding fragment comprises a variable heavy chain (VH) having the sequence of SEQ ID NO: 26 and a variable light chain (VL) having the sequence of SEQ ID NO: 30.

77. The kit of claim 66, wherein the antibody or antigen-binding fragment comprises a heavy chain having the sequence of SEQ ID NO: 33 and a light chain having the sequence of SEQ ID NO: 37.

78. The kit of claim 66, wherein the antibody or antigen-binding fragment comprises a heavy chain having the sequence of SEQ ID NO: 34 and a light chain having the sequence of SEQ ID NO: 38.

79. The kit of claim 66, wherein the antibody or antigen-binding fragment binds to human FOLR1 with a Kd of about 1.0 nM to about 10 nM.

80. The kit of claim 66, wherein the antibody or antigen-binding fragment binds to human FOLR1 with a Kd of about 0.5 nM to about 5 nM.


SEQUENCE LISTING (68 sequences)

SEQ ID NO: 1 - protein, 5 aa, Artificial Sequence
  VH-CDR1 muFR1-9
SFGMH

SEQ ID NO: 2 - protein, 17 aa, Artificial Sequence
  VH-CDR2 muFR1-9
YISSGSSTFYYADTVKG

SEQ ID NO: 3 - protein, 8 aa, Artificial Sequence
  VH-CDR3 muFR1-13
ELTGTFAY

SEQ ID NO: 4 - protein, 5 aa, Artificial Sequence
  VH-CDR1 muFR1-13
RYSVH

SEQ ID NO: 5 - protein, 16 aa, Artificial Sequence
  VH-CDR2 muFR1-13
MIWSGGNTDYNSVFKS

SEQ ID NO: 6 - protein, 10 aa, Artificial Sequence
  VH-CDR3 muFR1-13
FDGKVSWFAY

SEQ ID NO: 7 - protein, 5 aa, Artificial Sequence
  VH-CDR1 muFR1-53
DYDIS

SEQ ID NO: 8 - protein, 17 aa, Artificial Sequence
  VH-CDR2 muFR1-53
EIYPGSGRTYYNERFKG

SEQ ID NO: 9 - protein, 12 aa, Artificial Sequence
  VH-CDR3 muFR1-53
SYYYGTNSPFAY

SEQ ID NO: 10 - protein, 5 aa, Artificial Sequence
  VH-CDR1 muFR1-62
TYTMH

SEQ ID NO: 11 - protein, 17 aa, Artificial Sequence
  VH-CDR2 muFR1-62
YINPTSGYNNYNQKFKE

SEQ ID NO: 12 - protein, 11 aa, Artificial Sequence
  VH-CDR3 muFR1-62
GGAYGRRPVDY

SEQ ID NO: 13 - protein, 11 aa, Artificial Sequence
  VL-CDR1 muFR1-9
RASQSINNNLH

SEQ ID NO: 14 - protein, 7 aa, Artificial Sequence
  VL-CDR2 muFR1-9
YASQSIS

SEQ ID NO: 15 - protein, 10 aa, Artificial Sequence
  VL-CDR3 muFR1-9
QQSNSWPQVT

SEQ ID NO: 16 - protein, 11 aa, Artificial Sequence
  VL-CDR1 muFR1-13
KASQSVSNDVL

SEQ ID NO: 17 - protein, 7 aa, Artificial Sequence
  VL-CDR2 muFR1-13
YAYNRYS

SEQ ID NO: 18 - protein, 9 aa, Artificial Sequence
  VL-CDR3 muFR1-13
QQDHSSPFT

SEQ ID NO: 19 - protein, 11 aa, Artificial Sequence
  VL-CDR1 muFR1-53
RASQDISNYLH

SEQ ID NO: 20 - protein, 7 aa, Artificial Sequence
  VL-CDR2 muFR1-53
YTSRLQS

SEQ ID NO: 21 - protein, 9 aa, Artificial Sequence
  VL-CDR3 muFR1-53
QQGNSLPPT

SEQ ID NO: 22 - protein, 11 aa, Artificial Sequence
  VL-CDR1 muFR1-62
KASQNVGTNVA

SEQ ID NO: 23 - protein, 7 aa, Artificial Sequence
  VL-CDR2 muFR1-62
SASSRYS

SEQ ID NO: 24 - protein, 9 aa, Artificial Sequence
  VL-CDR3 muFR1-62
HQYNSYPYT

SEQ ID NO: 25 - protein, 117 aa, Artificial Sequence
  muFR1-9HCvar Variable heavy chain
QVQLVESGGGLVQPGGSRKLSCAASGFTFSSFGMHWVRQAPEKGLEWVAYISSGSSTFYYADTVKGRFTISRDNPKNTLFLQMTSLRSEDTAMYYCAKELTGTFAYWGQGTLVTVSA

SEQ ID NO: 26 - protein, 118 aa, Artificial Sequence
  muFR1-13HCvar Variable heavy chain
QVQLKESGPDLVAPSQSLSITCTVSGFSLSRYSVHWIRQPPGKGLEWLGMIWSGGNTDYNSVFKSRLNITKDNSKSQVFLKMNSLQTDDTAIYYCATFDGKVSWFAYWGQGTLVTVSA

SEQ ID NO: 27 - protein, 121 aa, Artificial Sequence
  muFR1-53HC Variable heavy chain
QVQLQQSGPELVRPGASVKMSCKASGYKFTDYDISWVLQRTGQGLEWIGEIYPGSGRTYYNERFKGKATLTADKSSNTVYMQLSSLTSEDSAVYFCASSYYYGTNSPFAYWGQGTTLTVSS

SEQ ID NO: 28 - protein, 120 aa, Artificial Sequence
  muFR1-62HC Variable heavy chain
QVQLQQSGAELARPGASVKMSCKASGYTFTTYTMHWVKQRPGQGLEWIAYINPTSGYNNYNQKFKEKATLTADKSSSTAYMQLTSLTSEDSAVYYCASGGAYGRRPVDYWGQGTSVTVSS

SEQ ID NO: 29 - protein, 109 aa, Artificial Sequence
  muFR1-9Lcvar Variable light chain
DIVLTQSPATLSVTPGDSVSLSCRASQSINNNLHWYQQKSHESPRLLIKYASQSISGIPSRFSGSGSGTDFTLSINSVETEDFGMYFCQQSNSWPQVTFGAGTKLELKR

SEQ ID NO: 30 - protein, 108 aa, Artificial Sequence
  muFR1-13LCvar Variable light chain
SIVMTQTPKFLLVSTGDRFTITCKASQSVSNDVLWYQQKPGQSPKLLIYYAYNRYSGVPDRFTGSGYGTDFTFTITTVQSEDLAVYFCQQDHSSPFTFGSGTKLEIKR

SEQ ID NO: 31 - protein, 108 aa, Artificial Sequence
  muFR1-53LC Variable light chain
DIQMTQTTSSLSASLGDRVTISCRASQDISNYLHWYQRKPDGTVKLLVYYTSRLQSGVPSRFSGSGSGTDYSLTISNLEQEDIATYFCQQGNSLPPTFGSGTKLEIKR

SEQ ID NO: 32 - protein, 108 aa, Artificial Sequence
  muFR1-62LC Variable light chain
DIVMTQSQKFMSISVGDRVSVTCKASQNVGTNVAWYQQKPGQSPKTLIYSASSRYSGVPDRFTGSGSGTDFTLTISNVQSEDLADYFCHQYNSYPYTFGGGTKLEIKR

SEQ ID NO: 33 - protein, 441 aa, Artificial Sequence
  muFR1-9HC Full-Length Heavy Chain Amino
QVQLVESGGGLVQPGGSRKLSCAASGFTFSSFGMHWVRQAPEKGLEWVAYISSGSSTFYYADTVKGRFTISRDNPKNTLFLQMTSLRSEDTAMYYCAKELTGTFAYWGQGTLVTVSAAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 34 - protein, 454 aa, Artificial Sequence
  muFR1-13HC Full-length heavy chain
QVQLKESGPDLVAPSQSLSITCTVSGFSLSRYSVHWIRQPPGKGLEWLGMIWSGGNTDYNSVFKSRLNITKDNSKSQVFLKMNSLQTDDTAIYYCATFDGKVSWFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 35 - protein, 445 aa, Artificial Sequence
  muFR1-53HC Full-length heavy chain
QVQLQQSGPELVRPGASVKMSCKASGYKFTDYDISWVLQRTGQGLEWIGEIYPGSGRTYYNERFKGKATLTADKSSNTVYMQLSSLTSEDSAVYFCASSYYYGTNSPFAYWGQGTTLTVSSAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 36 - protein, 444 aa, Artificial Sequence
  muFR1-62HC Full-Length Heavy Chain
QVQLQQSGAELARPGASVKMSCKASGYTFTTYTMHWVKQRPGQGLEWIAYINPTSGYNNYNQKFKEKATLTADKSSSTAYMQLTSLTSEDSAVYYCASGGAYGRRPVDYWGQGTSVTVSSAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 37 - protein, 215 aa, Artificial Sequence
  muFR1-9LC Full-length Light Chain
DIVLTQSPATLSVTPGDSVSLSCRASQSINNNLHWYQQKSHESPRLLIKYASQSISGIPSRFSGSGSGTDFTLSINSVETEDFGMYFCQQSNSWPQVTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - protein, 214 aa, Artificial Sequence
  muFR1-13LC Full-length Light  Chain
SIVMTQTPKFLLVSTGDRFTITCKASQSVSNDVLWYQQKPGQSPKLLIYYAYNRYSGVPDRFTGSGYGTDFTFTITTVQSEDLAVYFCQQDHSSPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 39 - protein, 214 aa, Artificial Sequence
  muFR1-53LC Full-length Light  Chain
DIQMTQTTSSLSASLGDRVTISCRASQDISNYLHWYQRKPDGTVKLLVYYTSRLQSGVPSRFSGSGSGTDYSLTISNLEQEDIATYFCQQGNSLPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 40 - protein, 214 aa, Artificial Sequence
  muFR1-62LC Full-length Light  Chain
DIVMTQSQKFMSISVGDRVSVTCKASQNVGTNVAWYQQKPGQSPKTLIYSASSRYSGVPDRFTGSGSGTDFTLTISNVQSEDLADYFCHQYNSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - protein, 257 aa, Artificial Sequence
  human FOLR1
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 42 - protein, 11 aa, Artificial Sequence
  FOLR1 Signature Peptide Sequence 1
TELLNVCMNAK

SEQ ID NO: 43 - protein, 5 aa, Artificial Sequence
  FOLR1 Signature Peptide Sequence 2
IAWAR

SEQ ID NO: 44 - protein, 8 aa, Artificial Sequence
  FOLR1 Signature Peptide Sequence 3
VLNVPLCK

SEQ ID NO: 45 - protein, 19 aa, Artificial Sequence
  FOLR1 Signature Peptide Sequence 4
CIQMWFDPAQGNPNEEVAR

SEQ ID NO: 46 - protein, 5 aa, Artificial Sequence
  VH-CDR1 muFRIHC2-1
NSYIH

SEQ ID NO: 47 - protein, 17 aa, Artificial Sequence
  VH-CDR2 muFRIHC2-1
WIYPESLNTQYNEKFKA

SEQ ID NO: 48 - protein, 13 aa, Artificial Sequence
  VH-CDR3 muFRIHC2-1
RGIYYYSPYALDH

SEQ ID NO: 49 - protein, 16 aa, Artificial Sequence
  VL-CDR1 muFRIHC2-1
KSSKSLLNSDGFTYLD

SEQ ID NO: 50 - protein, 7 aa, Artificial Sequence
  VL-CDR2 muFRIHC2-1
LVSNHFS

SEQ ID NO: 51 - protein, 9 aa, Artificial Sequence
  VL-CDR3 muFRIHC2-1
FQSNYLPLT

SEQ ID NO: 52 - protein, 122 aa, Artificial Sequence
  muFRIHC2-1 Variable heavy chain
QVQLQQSGPELVKPGASVRISCKASGYTFTNSYIHWVKKRPGQGLEWIGWIYPESLNTQYNEKFKAKATLTADKSSSTSYMQLSSLTSEDSAVYFCARRGIYYYSPYALDHWGQGASVTVSS

SEQ ID NO: 53 - protein, 114 aa, Artificial Sequence
  muFRIHC2-1 Variable light chain
SDVVLTQTPLSLPVNIGDQASISCKSSKSLLNSDGFTYLDWYLQKPGQSPQLLIYLVSNHFSGVPDRFSGSGSGTDFTLKISRVEAEDLGVYYCFQSNYLPLTFGGGTKLEIKR

SEQ ID NO: 54 - protein, 446 aa, Artificial Sequence
  muFRIHC2-1 Full-length heavy chain
QVQLQQSGPELVKPGASVRISCKASGYTFTNSYIHWVKKRPGQGLEWIGWIYPESLNTQYNEKFKAKATLTADKSSSTSYMQLSSLTSEDSAVYFCARRGIYYYSPYALDHWGQGASVTVSSAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 55 - protein, 220 aa, Artificial Sequence
  muFRIHC2-1 Full-length light chain
SDVVLTQTPLSLPVNIGDQASISCKSSKSLLNSDGFTYLDWYLQKPGQSPQLLIYLVSNHFSGVPDRFSGSGSGTDFTLKISRVEAEDLGVYYCFQSNYLPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 56 - protein, 118 aa, Artificial Sequence
  huMov19 Variable Heavy Chain
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 57 - protein, 112 aa, Artificial Sequence
  huMov19 Variable Light Chain version 1.00
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKR

SEQ ID NO: 58 - protein, 112 aa, Artificial Sequence
  huMov19 Variable Light Chain version 1.60
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 59 - protein, 15 aa, Artificial Sequence
  huMov19 Variable Light Chain CDR1
KASQSVSFAGTSLMH

SEQ ID NO: 60 - protein, 7 aa, Artificial Sequence
  huMov19 Variable Light Chain CDR2
RASNLEA

SEQ ID NO: 61 - protein, 9 aa, Artificial Sequence
  huMov19 Variable Light Chain CDR3
QQSREYPYT

SEQ ID NO: 62 - protein, 5 aa, Artificial Sequence
  huMov19 Variable Heavy Chain CDR1
GYFMN

SEQ ID NO: 63 - protein, 17 aa, Artificial Sequence
  huMov19 Variable Heavy Chain CDR2 - Kabat Defined
RIHPYDGDTFYNQKFQG

SEQ ID NO: 64 - protein, 10 aa, Artificial Sequence
  huMov19 Variable Heavy Chain CDR2 - Abm Defined
RIHPYDGDTF

SEQ ID NO: 65 - protein, 9 aa, Artificial Sequence
  huMov19 Variable Heavy Chain CDR3
YDGSRAMDY

SEQ ID NO: 66 - protein, 448 aa, Artificial Sequence
  huMov19 Heavy Chain Amino Acid Sequence
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 218 aa, Artificial Sequence
  huMov19 Light Chain version 1.00
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 218 aa, Artificial Sequence
  huMov19 Light Chain version 1.60
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC